(19) United States
(12) Patent Application Publication
(10) Pub. No.: US 2008/0124365 A1
(43) Pub. Date: May 29, 2008

(54) Innate immune response inducer

(75) Inventor: Dongho Kim, Seoul (KR)

Correspondence Address: JHK LAW, P.O. BOX 1078, LA CANADA, CA, 91012-1078, US

(73) Assignee: GENOLUTION PHARMACEUTICALS, INC., Seoul (KR)

(21) Appl. No.: 11/807,226

(22) Filed: May 24, 2007

Related U.S. Application Data
(60) Provisional application No. 60/803,056, filed on May 24, 2006.

(51) Int. Cl. A61K 31/70; C07H 21/00; A61K 39/00; A61P 37/00; C12N 5/06

(52) U.S. Cl. 424/278.1; 536/25.3; 435/375; 536/23.1; 514/44

(57) ABSTRACT

The present application describes a method of making concatenated RNA comprising, mixing a single stranded DNA template with a RNA transcription reagent and allowing transcription reaction to occur, and use of the RNA for anti-viral and anti-cancer purposes.

### CROSS-REFERENCE TO RELATED APPLICATIONS

The present application claims priority under 35 U.S.C. 119(e) to U.S. Provisional Application No. 60/803,056, filed May 24, 2006, which is incorporated by reference herein in its entirety.

### BACKGROUND OF THE INVENTION

1. Field of the Invention

The present invention relates to RNA molecules, including a mixture of single-stranded 5′-triphosphate containing concatenated RNA molecules, and their use for inducing interferon. The present application relates to methods of making the RNA molecules. The present invention also relates to methods for controlling interferon-beta induction by such molecules. The present invention also relates to anti-viral and anti-cancer usage of the RNA molecules, which utilize the interferon-beta induction pathway.

2. General Background and State of the Art

The early recognition of invasive pathogens by innate sensing is the most important defense mechanism of the immune system^{1,2}. Viral infection of mammalian cells results in activation of an innate immune response, which is mediated by interferons and cytokines that concomitantly inhibit viral replication³. Several Toll-Like Receptors (TLRs) have been identified in human and mice and are known to be expressed predominantly on cell types which are first to encounter intracellular pathogens¹. Double stranded RNA (dsRNA), including the synthetic analog poly inosine-poly cytosine (Poly IC), is known to activate TLR3, a cellular receptor that recognizes and initiates a potent anti-viral response by producing interferons⁴. Similarly, single stranded RNA (ssRNA), which includes the genomes of several viral RNA species, has been shown to interact with and activate TLR7 and TLR8⁵⁻⁸. dsRNA can be easily distinguished intracellularly as viral replication intermediates, however, it remains elusive how a simple ssRNA motif recognized by TLR7 and 8 is discerned by the cell to be either viral (exogenous) or endogenous in origin¹. Considering that TLRs are cell type specific and are present within unique localized intracellular compartments, recognition of dsRNA and/or ssRNA offers an important innate defense mechanism against viral infection, along with the recognition of CpG DNA motifs and/or envelope glycoproteins^{1,2}.

It was previously shown that short interfering RNAs (siRNAs) prepared by in vitro transcription using T7 RNA polymerase possess potent anti-Herpes Simplex Virus (HSV) activity that is mediated by the induction of type 1 interferons⁹ (US Patent Application Publication No. 2006/0178334). The anti-viral activity is dependent on the presence of a 5′ triphosphate motif on either strand of the siRNA duplex and the antiviral effects are reversed by simple treatment with calf intestinal phosphatase (CIP). Further studies showed the response is activated by any ssRNA transcribed by a phage polymerase⁹. It was hypothesized that a host defense system exists which protect the host cell from viral RNAs that may contain a 5′ triphosphate-motif. Experiments were performed with genomic RNA derived from the Influenza A virus. Influenza viral RNAs lack 5′ modifications since the virus-derived transcriptase is unable to modify the 5′ terminus of mRNAs in the cytoplasm¹⁰. Purified viral RNAs (FIG. 1A) derived from Influenza A virus were incubated in the presence or absence of CIP prior to transfection into HEK293 cells. After transfection, the cells were sequentially challenged by HSV harboring the EGFP reporter gene¹¹. When cells were pre-transfected with influenza viral RNA, they were protected from HSV infection in a manner that was dependent on pre-treatment with CIP (FIG. 1B). The CIP treatment is limited to the removal of the 5′ triphosphate and does not affect the integrity of the RNA⁹ (FIG. 1A). Whether the anti-viral effect is mediated by type 1 interferon induction was further investigated. The level of interferon a was determined from the media of cell culture that has been transfected with the viral RNA by ELISA (FIG. 1C). Consistent with the results from the anti-HSV response, the induction of interferon α is dependent on CIP treatment, which was shown to be augmented by prolonging exposure to CIP. To generalize this observation, the mouse cell line, NIH3T3 that stably expresses EGFP¹², was used. When this cell line was infected with Encephalomyocarditis virus (EMCV), the cytotoxic effect of the virus was measured by the loss of EGFP expression (FIG. 1D, the first vs. second row). The cytotoxic effect by EMCV was reduced when the cells were transfected with either T7 RNA or Influenza viral RNA prior to viral challenge (third and fourth rows). Clearly the antiviral activity is dependent on the presence of a 5′ triphosphate motif on introduced RNA (fourth vs. fifth rows) and this property is not limited to human cells. Others have recently published that ssRNA with 5′ triphosphate is a signal of viral infection and responding through RIG-1 binding^{13,14}.

Therefore, there is a need in the art to make a more effective anti-tumor or anti-viral compound.

### SUMMARY OF THE INVENTION

In one aspect, the present invention is directed to a method of making RNA comprising, mixing a single stranded DNA template with a RNA transcription reagent and allowing transcription reaction to occur. The template DNA may be about 19 to about 60 nucleotides long or about 24 to about 45 nucleotides long. Optionally, the DNA may be absent a promoter sequence. In this method, the DNA may have a partial-loop secondary structure, which allows for rolling circle type transcription. The produced RNA may have a length of at least 21 nucleotides, and the RNA may be concatenated.

In another aspect, the invention is directed to a method for inducing production of interferon in a cell, comprising introducing into the cell a composition comprising an effective amount of 5′-triphosphate concatenated RNA, wherein the RNA molecule induces the interferon. The interferon may be interferon-beta. The RNA molecule may have an anti-viral effect. And the RNA molecule may be introduced into the cell prior to viral infection. Further, the RNA molecule may inhibit viral infection.

In another aspect, the invention is directed to an isolated 5′-triphosphate RNA molecule having a length of at least 21 nucleotides, wherein the RNA molecule is absent a sequence that is complementary to a promoter sequence which is made in accordance with the methods described above. The RNA molecule may be concatenated.

In yet another aspect, the invention is directed to a method of inhibiting viral infection in a mammal comprising administering to the mammal a composition comprising an effective amount of the RNA molecule described above, which elicits an anti-viral response.

In still another aspect, the invention is directed to a method of killing tumor cells through apoptosis in a mammal, comprising administering to the mammal a composition comprising an effective amount of the RNA molecule described above.

In another aspect, the invention is directed to a method of inhibiting proliferation of cancerous cells in a mammal, comprising administering to the mammal a composition comprising an effective amount of the RNA molecule described above, wherein when the RNA molecule enters a normal cell, interferon-beta is secreted, and inhibits proliferation of nearby tumor cells.

In another aspect, the invention is directed to a method of activating innate immune response in a mammal, comprising administering to the mammal a composition comprising an effective amount of the RNA molecule described above.

These and other objects of the invention will be more fully understood from the following description of the invention, the referenced drawings attached hereto and the claims appended hereto.

### BRIEF DESCRIPTION OF THE DRAWINGS

The present invention will become more fully understood from the detailed description given herein below, and the accompanying drawings which are given by way of illustration only, and thus are not limitative of the present invention, and wherein:

FIGS. 1A-1D show that the 5′ triphosphate of RNA is a novel motif for stimulating the innate immune response. 1A) Total RNA was purified from influenza viral RNA and treated without (−CIP) or with (+CIP) calf intestinal phosphatase. 1B) HEK293 cells were transfected with no RNA (mock), influenza viral RNA without CIP treatment (Flu RNA −CIP), or the RNA with CIP treatment (Flu RNA +CIP) and sequentially challenged by EGFP-labeled HSV. The infection of virus was monitored by fluorescence microscopy. 1C). HEK293 cells were transfected with influenza viral RNAs without (second column) or with the CIP pretreatment for 10 (third) or 60 minutes (fourth column) and measured the induced level of interferon alpha using ELISA. 1D). NIH3T3 cells stably expressing EGFP were treated with no RNA (mock), 10 ng of T7 RNA (T7 RNA), 0.5 ug of influenza viral RNA without CIP pre-treatment (Flu RNA-CIP), and the viral RNA with CIP pre-treatment (Flu RNA +CIP). The next day (24 hours), cells were challenged with EMCV infection. On day 3, the viral infection mediated cytotoxic effect was monitored under light (the first panel) or fluorescence microscopy (the second panel).

FIGS. 2A-2C show that the nuclear derived nascent RNAs show an anti-viral activity that depends on the presence of the 5′tri-phosphate. 2A). Cytoplasmic and nuclear extracts were prepared from HEK293 cells and tested by Western blot for the cytoplasmic protein enolase or nuclear protein hnRNP H. 2B) Cytoplasm (the first lane) and Nuclear RNAs (second and third lanes) were purified from each extract and analyzed on a 1% agarose gel in the absence (second lane) or presence of CIP pre-treatment (third lane). 2C) HEK293 cells were transfected with each indicated RNAs and sequentially infected with EGFP-labeled HSV. The pictures were taken under fluorescence microscopy on day 3.

FIGS. 3A-3F show potent anti-viral activity of the inventive RNA transcribed from ssDNA oligos that are absent of promoter. 3A). The 5′ triphosphate containing ssRNA requires a size limit to show the anti-viral activity. Forty ug of each transcribed RNA was gel purified and transfected to NIH 3T3 cells in 24-well plate. After EMCV viral challenge the number of survived cells were counted in 24 hours. 3B). The anti-viral activity of the inventive RNA. NIH3T3 cells stably expressing EGFP was transfected with either mock or 1 ng/ml and sequentially infected with EMCV virus. The image was taken 24 hour of post infection. 3C). Transcriptional products using ssDNA oligos are RNA with repeated pattern. Left panel; agarose gel, M/ssDNA marker, 1; reaction used 35mer ssDNA, 2; the same reaction partially digested with RNase A. Right panel; the same sample was analyzed on the denaturing gel. 3D). The ssDNA dependent transcriptional reaction is influenced by high Mg²⁺ concentration. The reaction was carried out in different concentration of Mg in the presence of 30 nM of ssDNA oligo for overnight. 3E). The anti-viral activity is increased as more DNA oligos were used. The 20 ul of reaction mixture was diluted to 2,000 fold. Reaction mixture of 1 ul was transfected into NIH3T3 cell in 24 well plate before challenge by EMCV (MOI 0.5). Viable cells were counted next day as described in Materials and Methods. 3F). The anti-viral activity is increased as the incubation time of transcription reaction is increased. The same assay was used as described in FIG. 3E.

FIGS. 4A-4D show that RNA* is a potent anti-viral reagent. 4A). RNA* has less cytotoxic effects than poly IC. NIH3T3 cells expressing EGFP were transfected with the indicated amount of each RNA and monitored for anti-proliferation activity by measuring total EGFP activity in the extract after 4 days of transfection. 4B). RNA* has more potency than poly IC in anti-viral activity. The indicated amount of each RNAs was transfected before infection with 0.5 MOI of EMCV. After 4 days of infection, the survived cells were counted by the level of EGFP expression. Each EGFP value was normalized to the RNA transfected cells without viral infection. The data represent the means of three independent assays. 4C). The same assays in FIG. 4B. were performed six days later to compare the anti-viral activity over a longer term. The data represent the means of three independent assays. 4D). The anti-viral activity of RNA* is more than a specific siRNA. HEK 293 cells were transfected with the indicated RNA materials and sequentially challenged by polio virus. The viability of cells was determined by cell counting in 24 hours. The approximate molar concentration was determined based on speculated molecular weight of RNA* shown in FIG. 3C.

FIGS. 5A-5C show that the anti-viral activity of RNA* is the result of synergistic effects of the presence of 5′ppp and its RNA structure. 5A) RNA* was treated with the indicated enzyme and analyzed on an agarose gel. 5B) The treated RNAs were transfected to NIH3T3 cells for the anti-viral assay. The survival % is average of two independent assays. 5C) Long dsRNAs were in vitro transcribed and tested for the anti-viral assay in the absence or presence of CIP treatment. The data is average of two independent assays.

FIGS. 6A-6C show RNA* mediated innate immune response requires TLR3. 6A). RNA* induces the expression of TLR3. TLR3 is upregulated by poly IC as well as by T7 RNA. Total RNA of NIH3T3 cells were harvested after transfection with no RNA, T7 RNA, and poly IC. The expression level of TLR3 was compared based on microarray data (data not shown). TLR7 and beta-actin were used as internal controls. 6B). TLR3 is required for the antiviral activity of poly IC and RNA*. On day 1, 20 nM of each siRNA was transfected. On day 3, RNA* (5 ng) or poly IC (250 ng) was transfected to the cells. On day 4, the treated cells were infected with HSV. On day 5, the viability of cells was determined using MTA assay. The viability of cells was determined as relative ratio of samples treated with scrambled siRNA. 6C). TLR3 is required for RNA* mediated innate immune response. MRC-5 cells were pre-incubated in the presence of anti-TLR2 or TLR3 antibodies and transfected in the presence of the indicated RNAs. The secreted interferon beta in the media was determined by ELISA in three independent assays.

FIG. 7 shows anti-proliferation activity of media used to grow cells transfected with RNA*. The media was taken from the transfected plate and diluted as indicated and tested for anti-proliferation activity.

### DETAILED DESCRIPTION OF THE PREFERRED EMBODIMENTS

In the present application, “a” and “an” are used to refer to both single and a plurality of objects.

As used herein, “anti-viral” means the lack of expression of viral protein and infection.

As used herein, “RNA*” refers to the 5′-phosphorylated RNA made using the rolling circle mechanism from a DNA template as discussed in the present application, infra. The RNA mixture comprises monomeric or multimeric forms of the transcribed RNA. The multimeric forms are concatenations of the monomers.

As used herein, “transcription reaction buffer” or “transcription reaction reagent” refers to any self-contained reagent system which contains the necessary enzymes and other factors to effectuate transcription of RNA from the DNA template. Nucleotides, enzymes and other factors may be stored separately, but the necessary ingredients are contacted together when the transcription reaction is to occur. Transcription reagents may be commercially purchased.

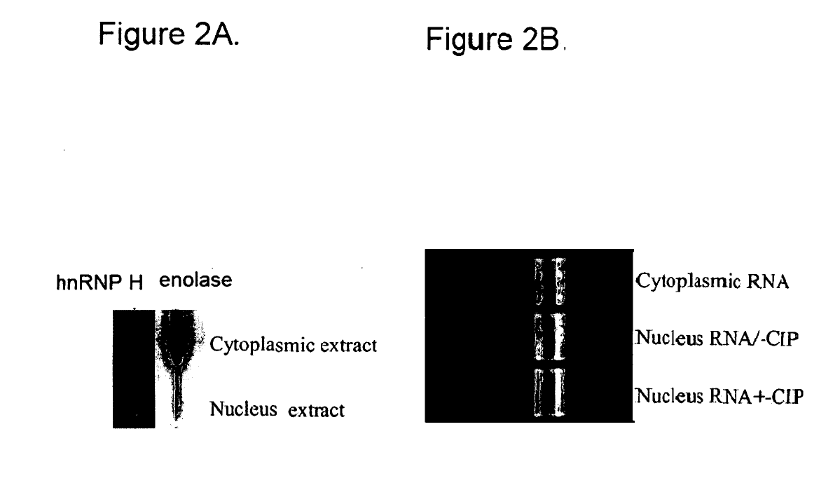

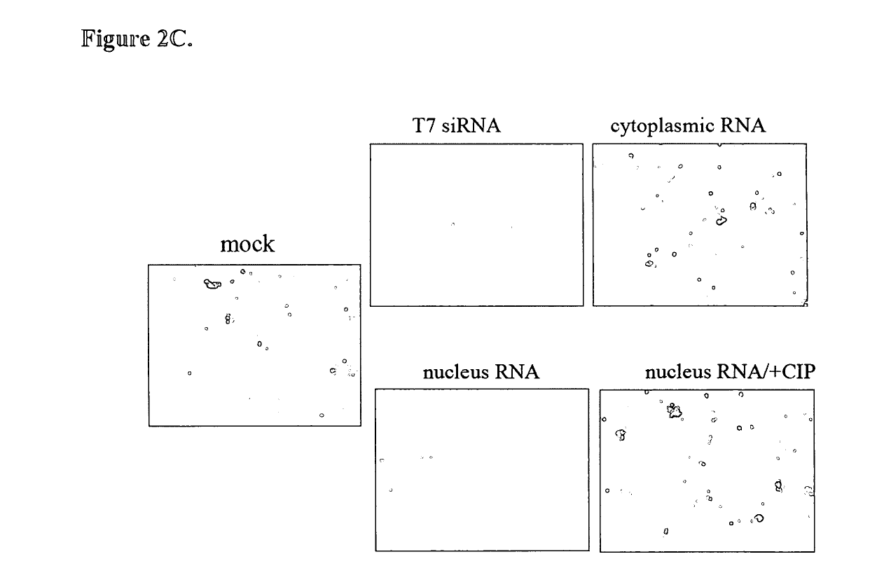

Since any 5′ triphosphate group present on RNA may induce an innate immune response, there is the distinct possibility that endogenous cellular RNAs can be potentially self-immunogenic. Although all nascent transcripts in the nucleus may harbor a 5′ triphosphate label, these are likely to be removed through a capping process before export to the cytoplasm 6. A cytoplasmic and nuclear extract of HEK293 cells was prepared and confirmed by Western blot using either nucleus (hnRNP H)¹⁷ or cytoplasm specific proteins (enolase)¹⁸ (FIG. 2A). The majority of enolase was present in the cytoplasmic extract. Conversely the nuclear protein, hnRNP H, was present in the nuclear extract. The RNA was purified from each extract to determine their immunogenic characteristics (FIG. 2B). Nuclear and cytoplasmic fractionated RNAs were transfected into HEK293 cells prior to challenge with HSV (FIG. 2C). No anti-HSV activity was detected using the RNA derived from the cytoplasmic fraction. Interestingly, the nuclear-derived RNAs showed an anti-viral response that could be abrogated by prior treatment with CIP (FIG. 2C). These data clearly indicate that two types of RNAs separated by the nuclear membrane have different immunogenic characteristics through the presence of a 5′ triphosphate motif. This possibly implies that cells have adopted their antiviral defense strategy based on the biological principal that all cytoplasmic RNAs lack a 5′ triphosphate following capping and methylation. Thus, cells may mistakenly sense transfected 5′-triphosphate-contained RNAs in the cytoplasm as incoming viral RNAs, thereby activating the innate immune system as a defense mechanism.

A comprehensive profile of gene expression by double-stranded RNA has been previously undertaken¹⁹. However, to characterize the signaling pathways elicited by 5′-triphosphate labeled RNA, NIH3T3 cells were transfected with either poly IC or T7-generated RNA (T7 RNA) and their relative gene expression profiles were compared using a murine oligonucleotide microarray. To determine the optimum time taken for gene expression, RNA was extracted at three different time points: 4, 8, and 16 hours following transfection with T7 RNA. Unlike the early response by Poly IC¹⁹, none of the changes were detected until after 16 hours, indicating that the T7 RNA mediated response is slower than Poly IC (data not presented). When the cells were transfected with T7 RNA, 86 genes were upregulated among 16,261 elements by a three-fold threshold (data not shown). In a parallel experiment, 229 and 12 genes were up or down-regulated respectively in poly IC-transfected cells. Interestingly, all 86 genes upregulated by T7 RNA transfection were also upregulated by poly IC. These data indicate that the signal pathway by T7 RNA overlaps with that of poly IC (data not shown). Activation of additional genes by poly IC indicates that poly IC affects a greater variety of genes (data not shown). Combined, all the T7 RNA mediated signal pathways are unique response through RIG-1 binding^{13,14}.

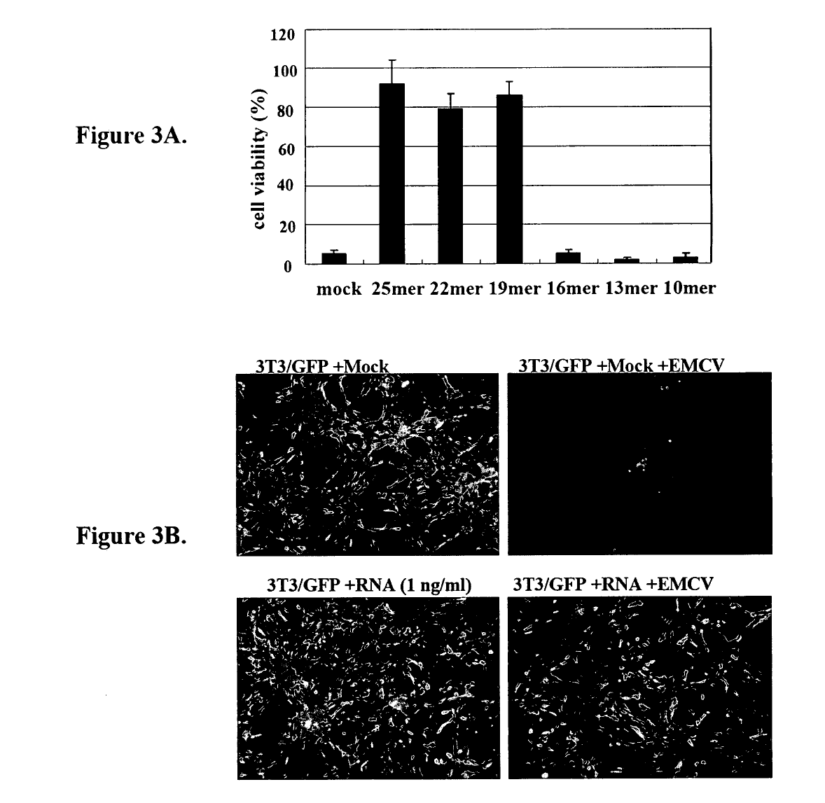

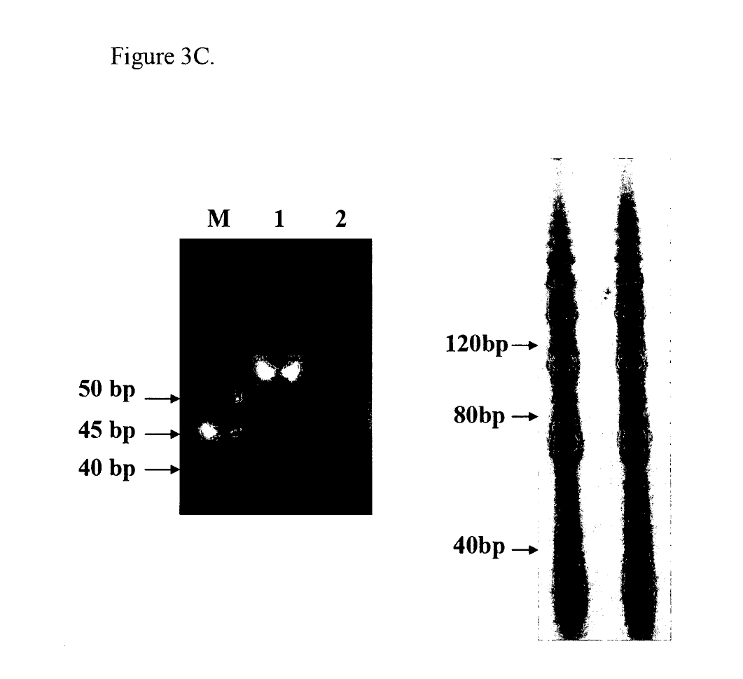

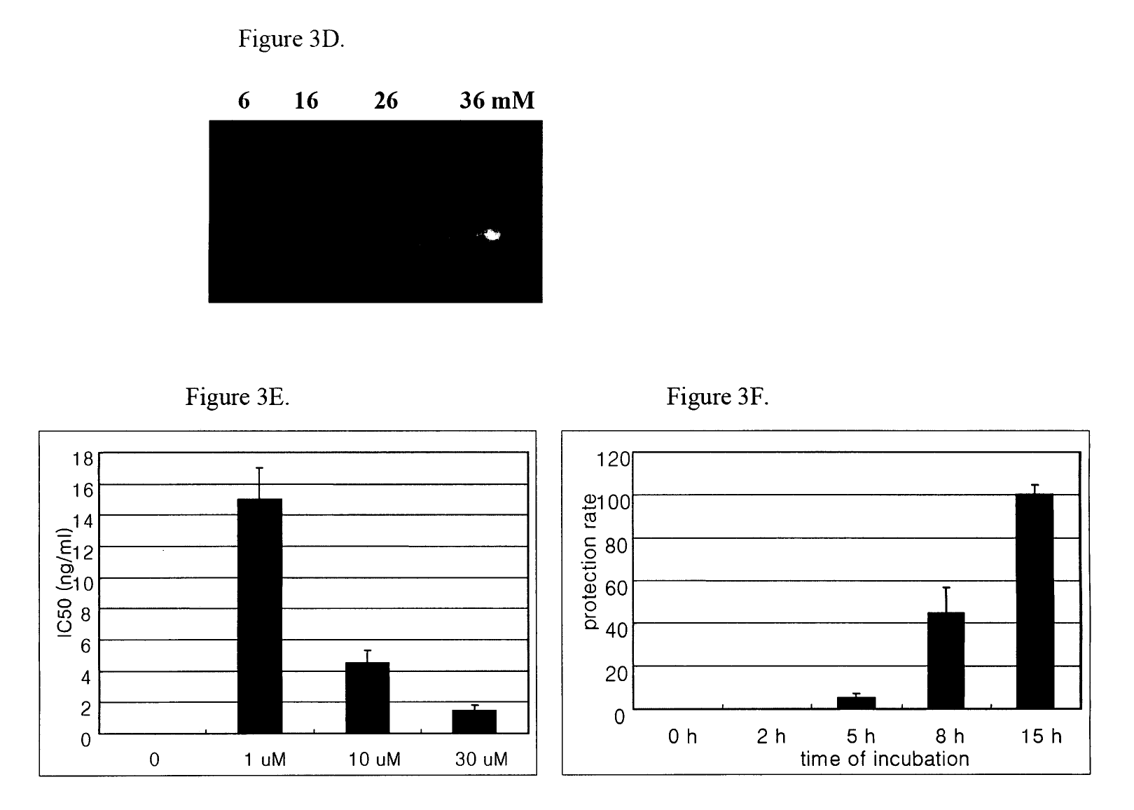

The minimal length of in vitro transcribed RNA for induction of the response in monocytes was determined as 19 mer¹⁴. The size limit of ssRNA molecule was tested using HEK293. Coincidentally, anti-viral activity was not detected when the ssRNA was shorter than 19 nt (FIG. 3A). This may indicate that the “sensing molecule” for 5′ triphosphate-containing ssRNA is limited by the length of the RNA sequence. Moreover, the size limit is not cell type specific. Several sequences of 20 nt ssRNA were transcribed and tested for their anti-viral activity. None of significant difference in antiviral activity was observed in each ssRNA molecules (data not shown). The antiviral activity is further decreased if part of dsDNA is disrupted (Table 1). Surprisingly, up to 100 fold increase of activity was detected if a pair of DNA oligos of the same polarity was used (Table 1 and FIG. 3B). A similar efficacy was observed when the polarity of both DNA oligos was reversed (Table 1). Since the two equi-polar DNA oligos cannot form a double helix, it was speculated that the anti-viral activity might be acquired even in the presence of ssDNA oligos. When the transcription reaction is performed in the presence of each single stranded DNA oligo, the same activity was obtained (Table 2). If partially duplexed DNA is presented, the observed anti-viral activity was decreased, indicating that the anti-viral reaction prefers ssDNA oligos (Table 3). Several tested oligos showed the same activity indicate that the activity is not sequence dependent (Table 3). The reaction mixture was analyzed on, agarose and denature gel to show the product is an RNA with repeated sizes (FIG. 3C).

Since not all ssDNA can be transcribed in this method (data not shown), a specific requirement for the reaction was searched (Table 4). When the ssDNA templates have either a loose structure such as a noose structure or a tight structure like a hairpin structure, the transcription reaction did not occur. It shows that partial structures of ssDNA templates are essential for transcription as well as its anti-viral activity. The sequence of RNA product was determined by RACE. In both tested cases, the RNA turned out to be the monomer or multimer depending on the band size (Table 5). It is interesting that the first three nucleotides in the 3′ end are not transcribed in the monomer. However the sequences are faithfully transcribed in next round of transcription of all sequenced multimer.

A detailed investigation for the optimum activity was further investigated. When the reaction was performed in the presence of 6 mM of Mg²⁺, which is the standard condition for in vitro transcription, the RNA was not made (FIG. 3D). However, product formation increased as the Mg²⁺ concentration was increased. Moreover, the activity was further increased with more amount of ssDNA in the reaction (FIG. 3E) and longer incubation (FIG. 3F) than the standard transcriptional reaction. The inventively characterized RNA reaction product was denoted as RNA*.

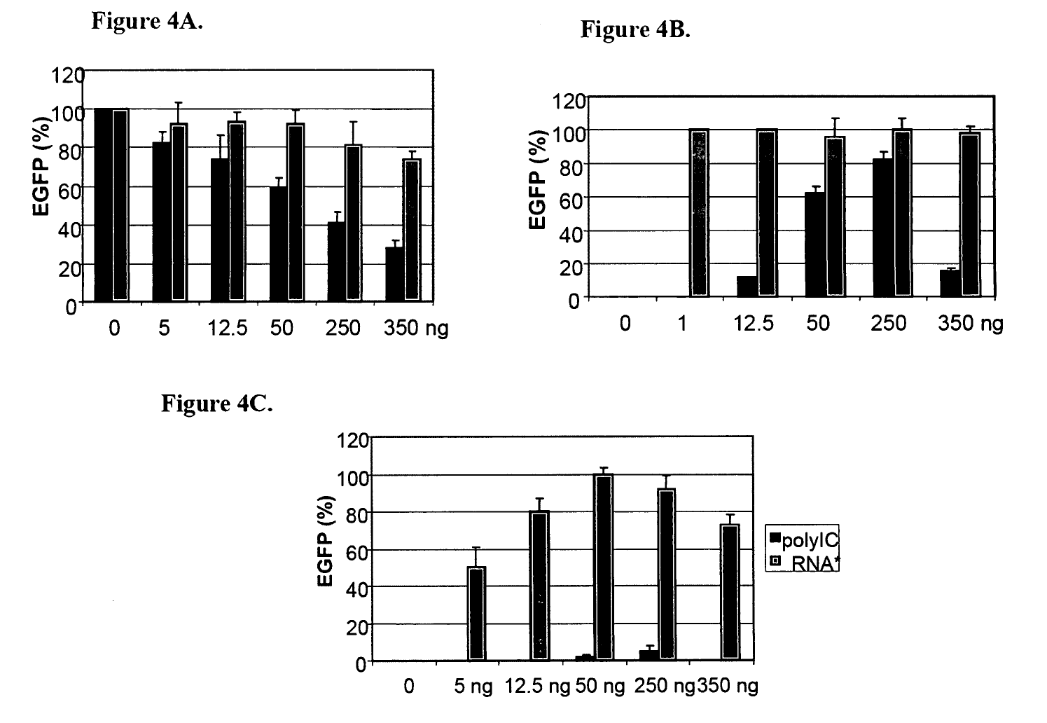

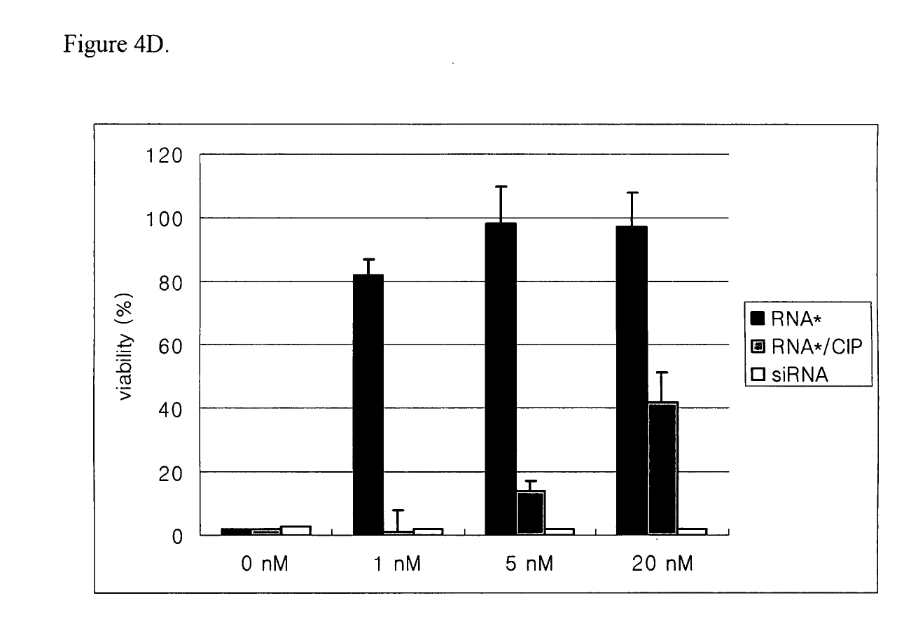

Poly IC is presently being used as potential application for anti-viral^{20,21} or cancer treatment²²⁻²⁵. However, cellular toxicities have been reported, which include the activation of apoptosis and/or insulitis^{19,26,27}. The RNA* was tested as a potential anti-viral agent and compared to the effects of poly IC. NIH3T3 cells were transfected with each RNA and cellular proliferation was measured thereafter (FIG. 4A). In contrast to the strong toxic effect elicited by poly IC, RNA* showed markedly reduced anti-proliferation activity. The anti-viral assay was performed as described in FIG. 3A using the NIH3T3 cells stably expressing EGFP. Following transfection of RNA* or Poly IC, the cells were challenged by EMCV virus to determine the anti-viral protective properties of the introduced RNA. These assays were standardized to the samples that were transfected with RNAs without subsequent viral infection. Cells transfected with 1 ng RNA* showed complete protection from ECMV infection, which was markedly more effective than the protection conferred by poly IC-transfected cells (FIG. 4B and data not shown). RNA* prevented viral infection at a concentration hundred-fold less than that used for poly IC. Interestingly, the protection was reduced when more than 250 ng of poly IC was used. The same assay was performed to determine the effects of long-term protection (FIG. 4C). Although none of the poly IC-transfected samples showed any protection after day 6, RNA* transfected cells showed significant protection. Consistent with the results for poly IC in FIG. 4B, the protection profile of RNA* also displayed a “bell shaped” distribution, suggesting an optimum ligand concentration for the best protection efficacy. These data indicate that when compared to poly IC, RNA* is a less toxic and a more potent anti-viral agent. Moreover, the anti-viral effects last longer and can be elicited using hundred-fold less amount of RNA. The similar trend was confirmed using a different cell line (MRC5) challenged by HSV (data not shown).

Among several potential therapeutic applications for siRNAs, their use as potential antiviral agents has been widely described^{28,29}. It has been previously reported that pre-treatment of cells with a siRNA to the poliovirus genome reduces viral titers²⁹. The same sequence of siRNAs or RNA* was used, and then the cells transfected with these RNAs were tested by challenging them with poliovirus infection (FIG. 4D). Less than 1 nM of RNA* showed very potent protection. In contrast, no anti-poliovirus activity was detected with the siRNAs until treatment with 300 nM siRNA (FIG. 4D and data not shown). When the RNA* was treated with CIP, the antiviral activity was abrogated in concentration dependent manner.

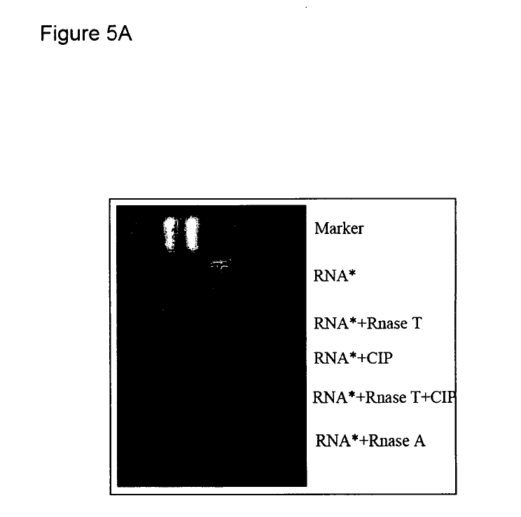

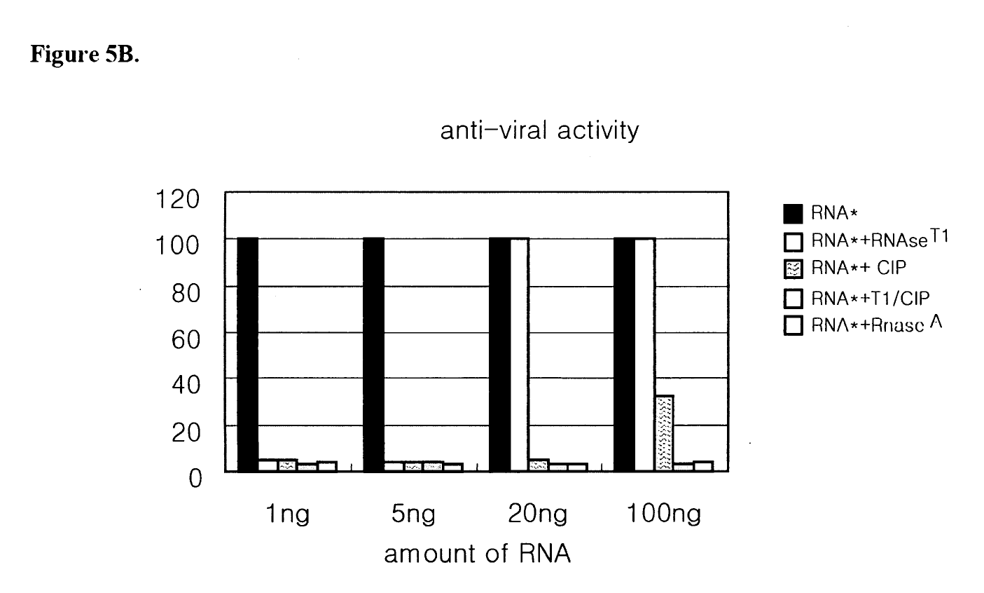

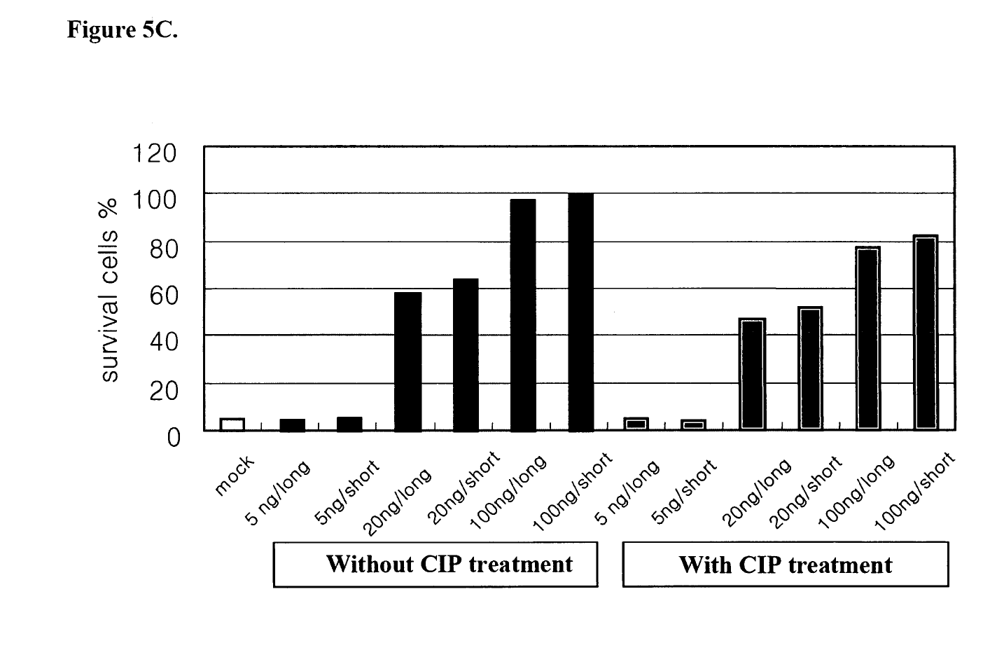

To understand why RNA* is much more potent than polyIC in the anti-viral response, cellular response was tested using microarray analyses. Consistent to cellular response (FIG. 4A to C) the response by RNA* is different to that of polyIC (data not shown). Unlike the delayed response of T7 RNAs made from T7 promoter, RNA* shows changes in even after 2 hour treatment. An individual contributing factor for the anti-viral activity of RNA* was further analyzed. Each RNA was treated with specific enzymes and the reaction was confirmed on the gel (FIG. 5A). RNA* produced from the ssDNA template containing 3′ GTA (Table 5) was incubated in the presence of RNAse T1 to disrupt the long dsRNA structure. The reaction converts the repeated RNA molecules to shorter form by cleavage of 3′-phosphate of G residue in ssRNA region (FIG. 5A). When the cleavage product is transfected to the cells the activity is lost in lower dosage (FIG. 5B). However, the activity is recovered when more RNA is used. In contrast, less activity was recovered for the CIP treated samples that were tested. It indicates that the effect of RNA* is mediated by the synergistic effect of the presence of 5′ ppp and the nature of its RNA structure. When these two factors are considered, the 5′ppp effect is more important than the structure of the RNA.

If the advantageous effect of RNA* is the result of a combination of the presence of 5′ppp and the length of ssRNA, similar effects are expected when in vitro transcribed long dsRNA is used. Partial or full length of EGFP genes were cloned under pBluescript vector and transcribed in both directions for long dsRNA. Each RNA was tested for the anti-viral activity in the presence or absence of CIP treatment (FIG. 5C). Heterogeneous RNA* was not used in this comparison because the molar calculation to obtain the same amount was not feasible. When the cells are treated with dsRNA the cells show an anti-viral activity. However the activity did not disappear even after the CIP treatment. This is in contrast to the results obtained with RNA*. The major factor contributing to the effects of the transcribed dsRNA can be attributed to dsRNA structure instead of the presence of the 5′ ppp motif. The potent anti-viral activity of RNA* can be explained by the combinational effect of 5′ ppp and short repeated ssRNAs instead of long dsRNA with the complete complementary structures.

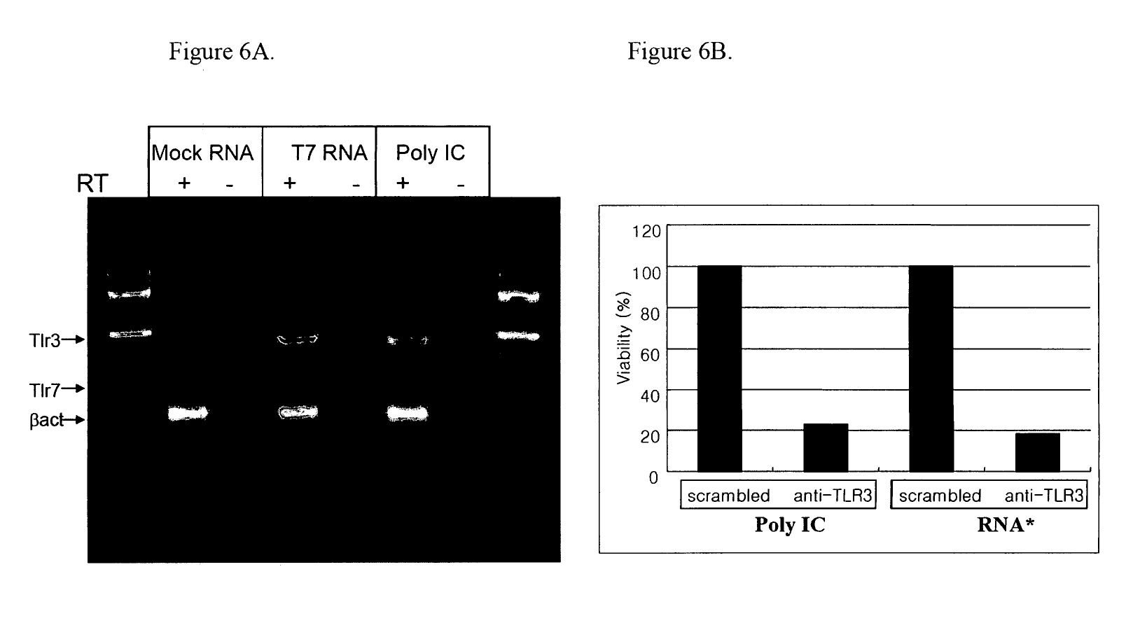

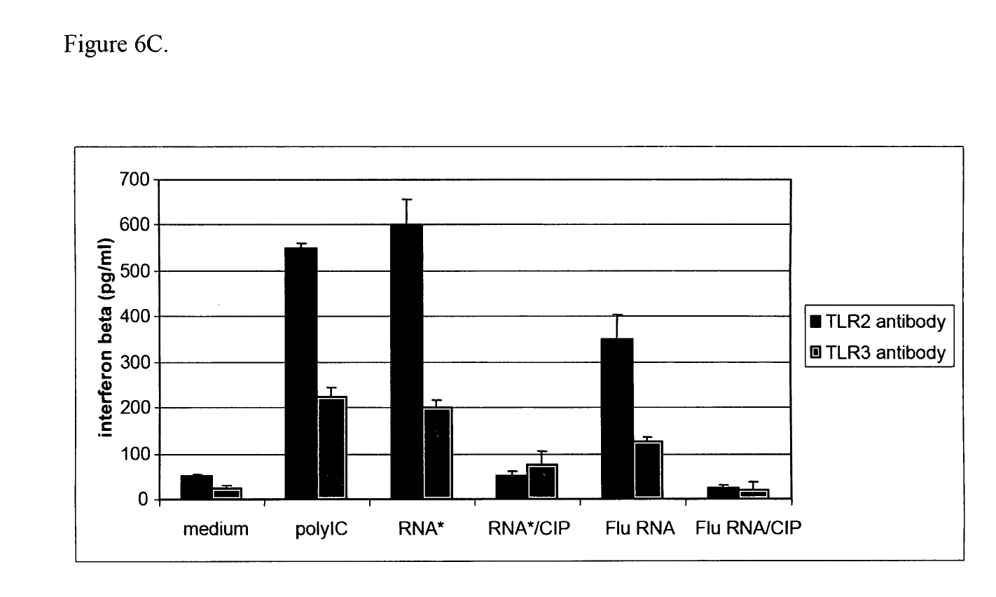

Each TLR detects pathogens that present detectable motifs termed pathogen-associated molecular pattern (PAMP), which are displayed on the surface of the invading organisms^{1,2,30}. To define the receptor for RNA*, the expression of Toll Like Receptors were compared in microarray data generated from RNA-transfected NIH3T3 cells. The microarray results showed that RNA*, including poly IC, induces TLR3 expression which was further confirmed by RT-PCR (data not presented and FIG. 6A). To determine if TLR3 is involved in the RNA* mediated response, the anti-viral activity of RNAs were tested with cells pretreated by an anti-TLR3 siRNA (FIG. 6B). As was known by previous studies, the poly IC mediated anti-viral activity is abrogated with pre-treatment of an anti-TLR3 siRNA³¹. Notably, the same pattern of response was observed when using RNA*, indicating that RNA* also requires the TLR3 receptor. Recognition of RNA* by TLR3 was additionally confirmed by a functional inhibition assay using appropriate antibodies. The IFN-β production of poly IC is known to be inhibited by an anti-TLR3 mAb in a human lung fibroblast cell line, MRC-5, which expresses TLR3 on the cell surface³⁰. When RNA* or influenza viral RNA was transfected into these cells, expression levels of interferon beta increased similarly to Poly IC-treated cells (FIG. 6C). The induction was inhibited when the cells were pre-incubated in the presence of an anti-TLR3 antibody, but not by an anti-TLR2 antibody. When both RNAs were treated with CIP, IFN-β induction was significantly reduced. This indicates that the inventive RNA-mediated response is channeled through TLR3.

The effector molecule to the anti-viral activity was investigated. After NIH3T3 cells were transfected with RNA* the media was aliquoted and tested for the activity. The culture media show clear anti-viral activity. The media was mixed with individual cytokines antibodies. Unlike other antibodies, the antibody against interferon beta totally blocked the anti-viral activity (data not shown). When the human interferon beta is cloned and overexpressed in 293 cell the anti-viral activity was observed in the cell culture media (Table 6). Combined all the anti-viral activity is caused by interferon beta induced by treatment of RNA*.

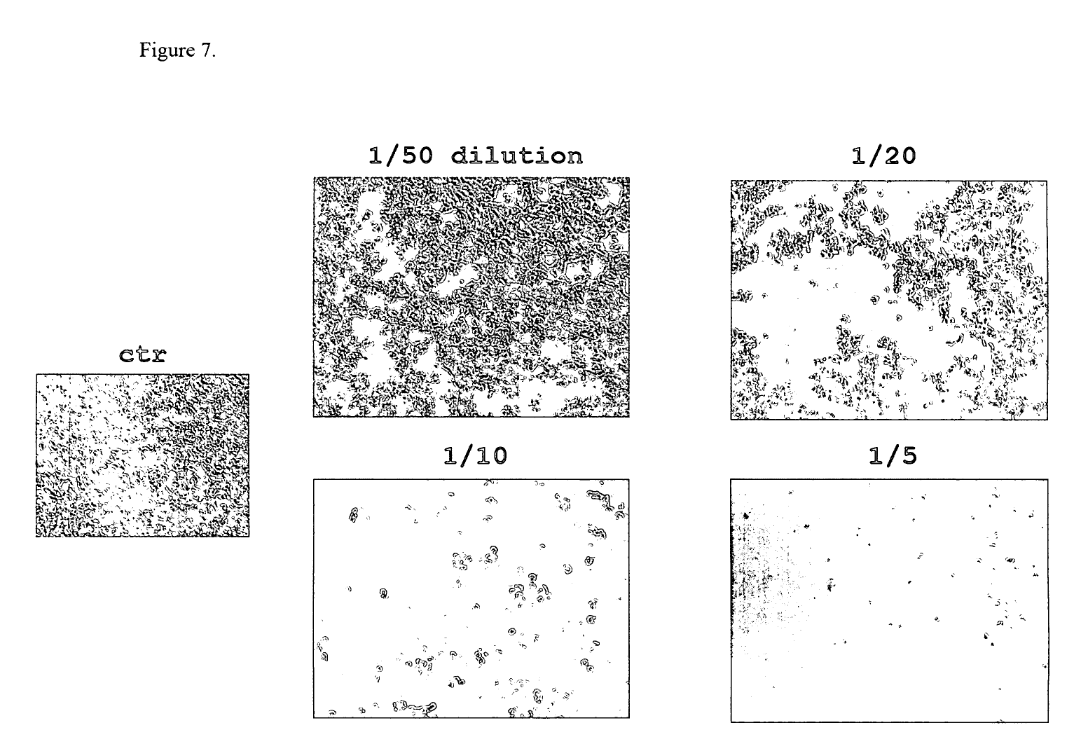

To see an effect to cancer cells culture media from cells transfected with RNA* was collected and added to cancer cells after dilution (FIG. 7). There was an anti-proliferation activity in a concentration dependent manner. To determine the effector molecule same approaches were made using several cytokine antibodies. When interferon beta antibody was used the activity was completely blocked (data not shown). Together with anti-viral activity the anti-proliferation activity is also attributed to interferon beta.

When RNA* is transfected into different cell types each shows unique response (Table 7). All tested cancer cell lines show clear apoptosis in less than 24 hours. However, partial cell death was detected after 72 hours in all non-cancerous cells. To determine any trans-acting factor with an anti-cancer effect in the RNA* treated media each sample was taken and mixed to MCF7 culture. All media from cancer cells show no anti-proliferation activity. Potent anti-proliferation activity was detected all samples from non-cancerous cells. RNA* has a dual activity based on cell types. If cancerous cells are transfected RNA* induces radical apoptosis in less than 24 hours. In non-cancerous cells secretion of interferon beta and delayed apoptosis was detected. If RNA* is transfected to cancer stricken individual it can be beneficial in two different ways. If RNA* is reached to cancer cells a cell death can be induced. If the molecule is transfected to normal cells it can induce interferon beta secretion that initiates the death of nearby cancer cells.

It is known that siRNAs made by in vitro transcription have an anti-viral effect, which is dependent on the presence of a 5′ triphosphate RNA motif⁹. The innate immune response is designed to protect invaded cells from viral genomic RNAs containing unmodified 5′ ends. This principle was further outlined by findings that nuclear-derived RNAs (with 5′ triphosphate ends) are immunogenic but not cytoplasmic-derived RNAs. Cells have developed a defense system based on the principle that nascent RNAs in the nucleus are processed to remove the 5′ triphosphate group before export to the cytoplasm. Appearance of RNAs containing a 5′ triphosphate motif in the cytoplasm is recognized as a “danger signal”, possibly indicating the invasion of a pathogenic threat. Similar studies have been published recently^{13,14}. They showed the 5′ppp containing RNA is ligand of RIG-1 for further anti-viral responses. One group also have shown that NS1 selectively targets RIG-1 rather than dsRNA during influenza viral infection¹³. By interference of NS1 the RIG-1 recognition for influenza ssRNA genome is suppressed. When mouse mRNA, total mammalian RNA, and tRNA were tested, they did not elicit IFN response and suggested that 5′ triphosphate is specific for viral RNA¹³. Data presented herein indicate that the response can be elicited either by the viral RNA or purified nuclear RNA but not by cytoplasmic RNA (FIG. 2C). Since total amount of cellular RNA is much abundant the uptake of nuclear RNA may be blocked in the transfection because of the limited amount of liposome.

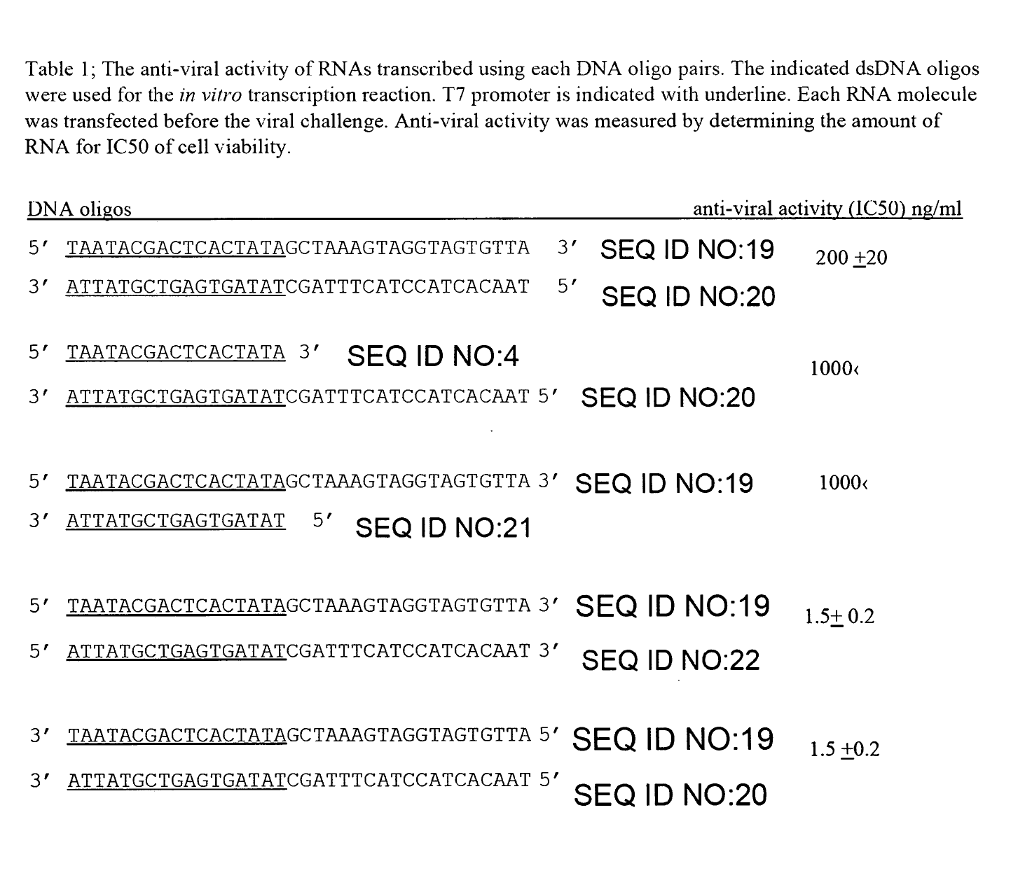

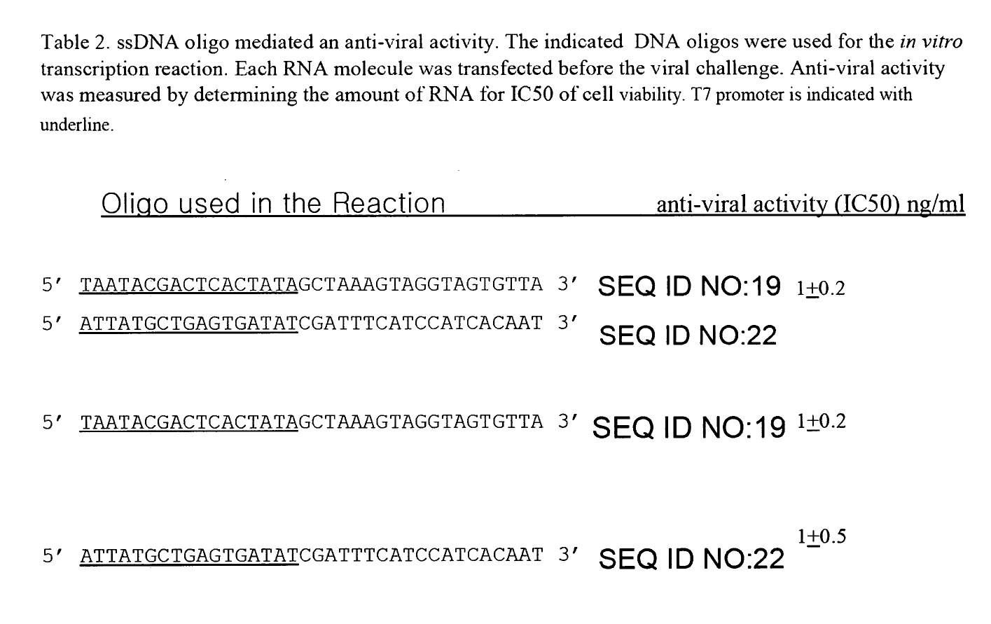

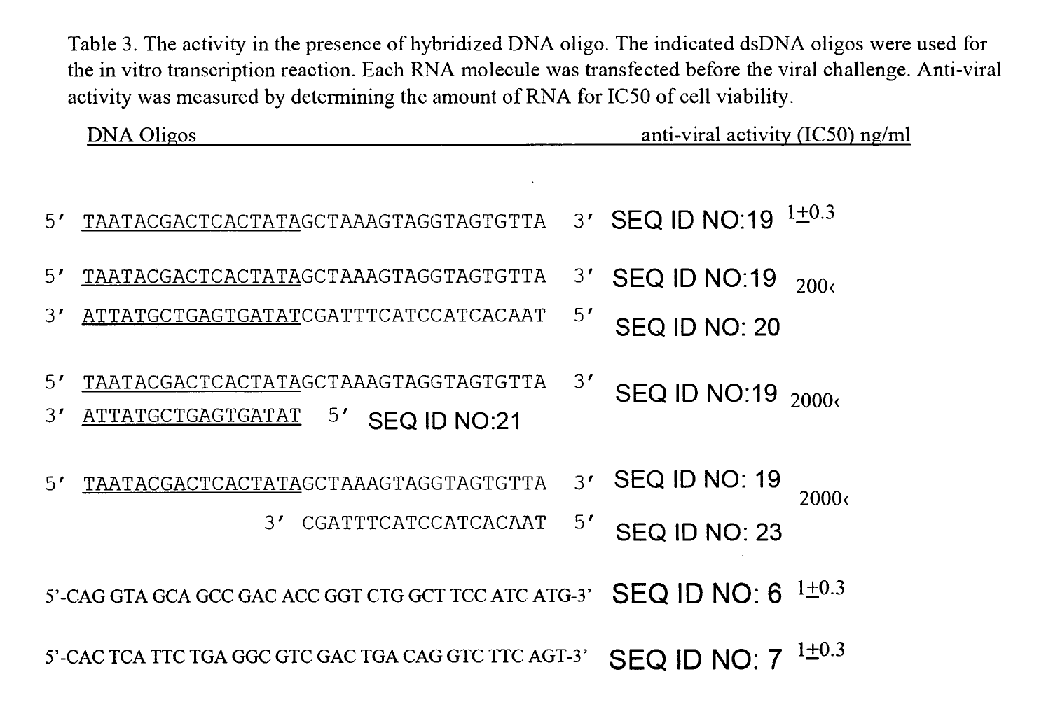

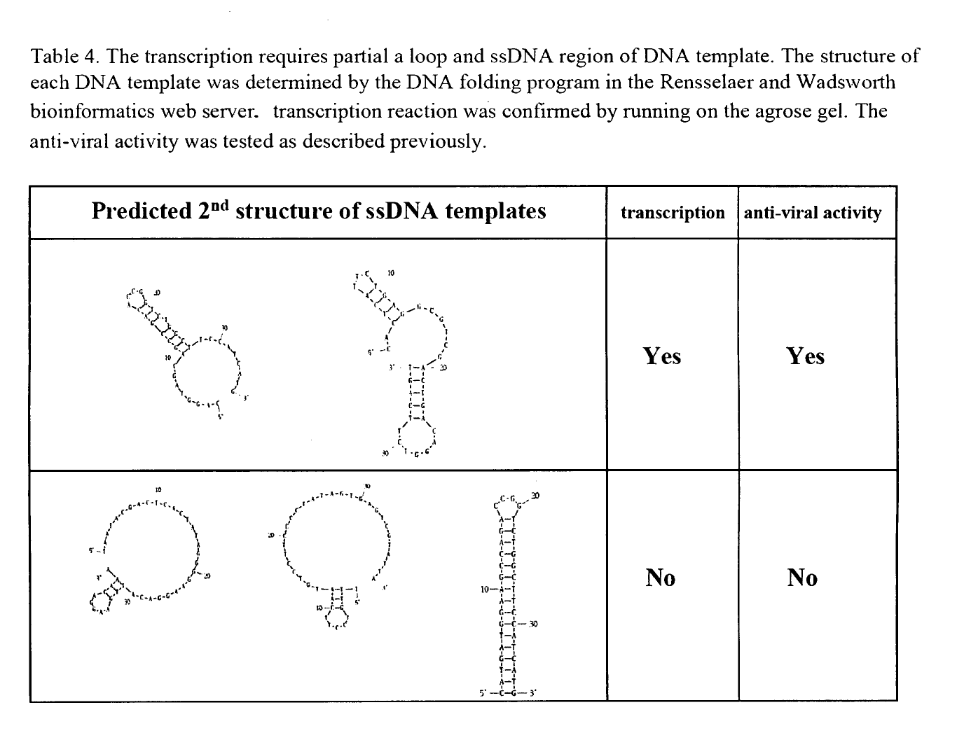

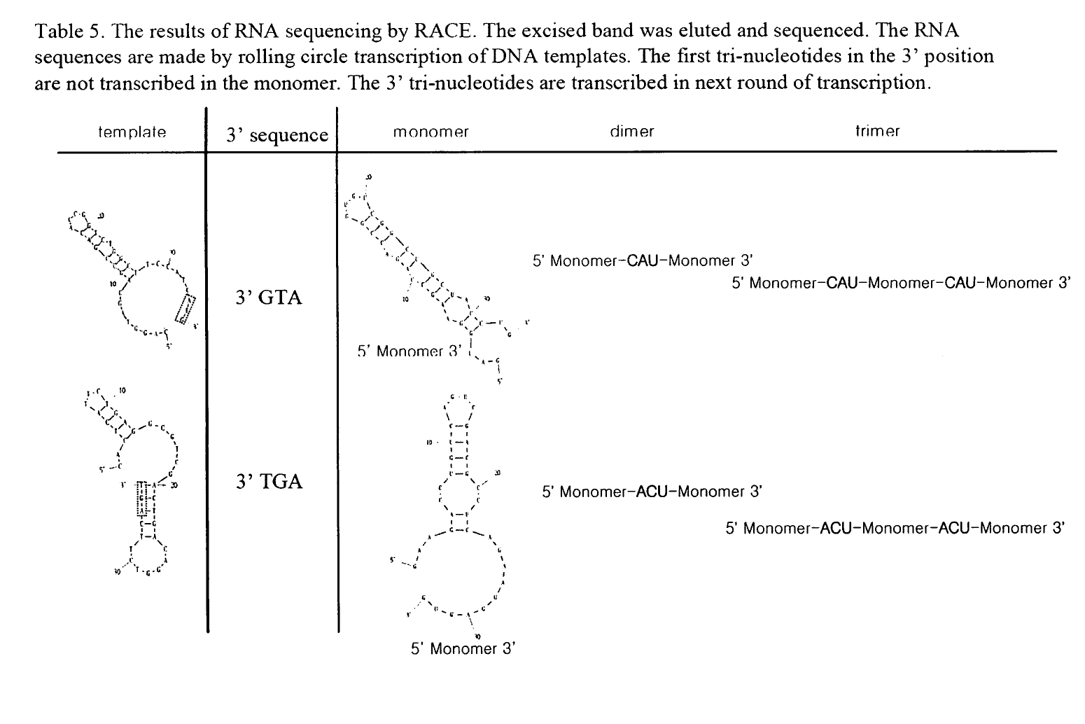

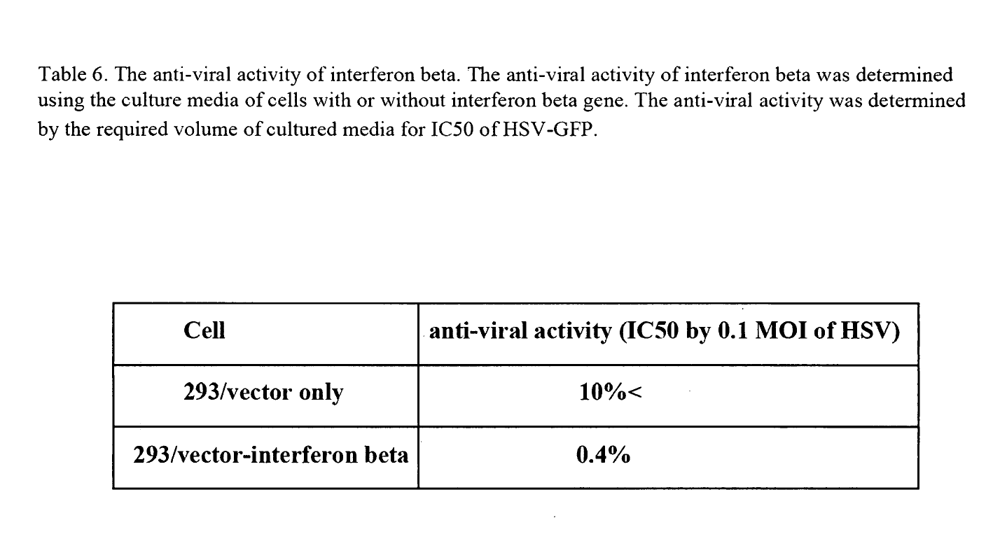

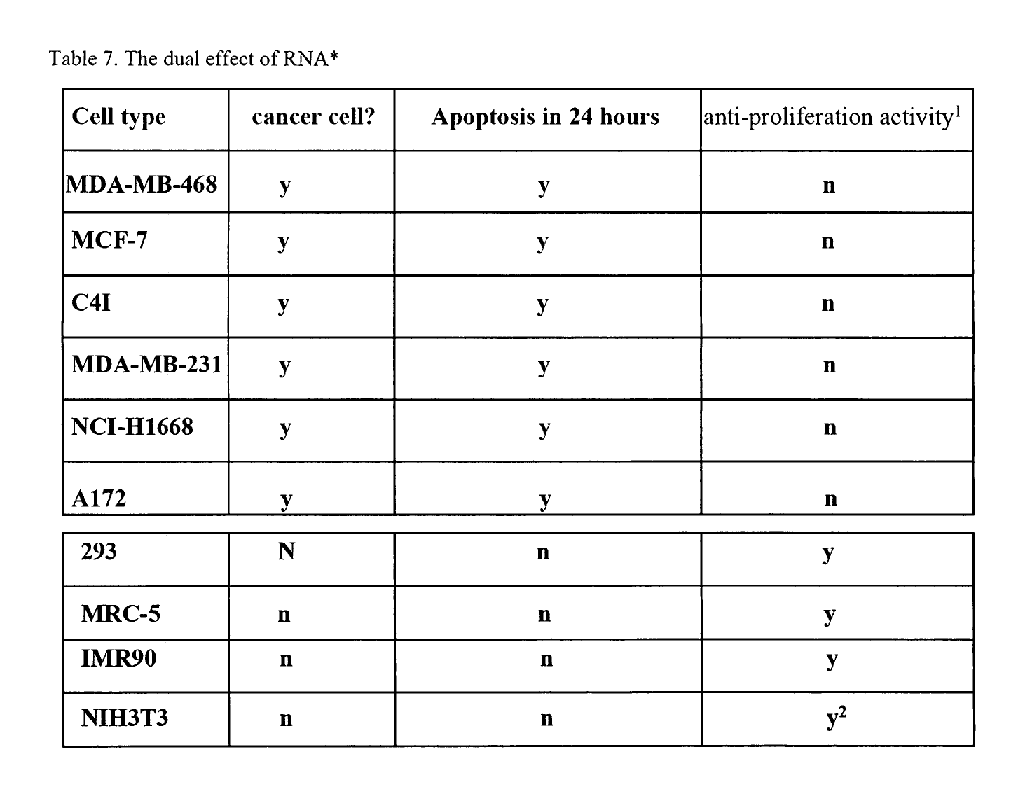

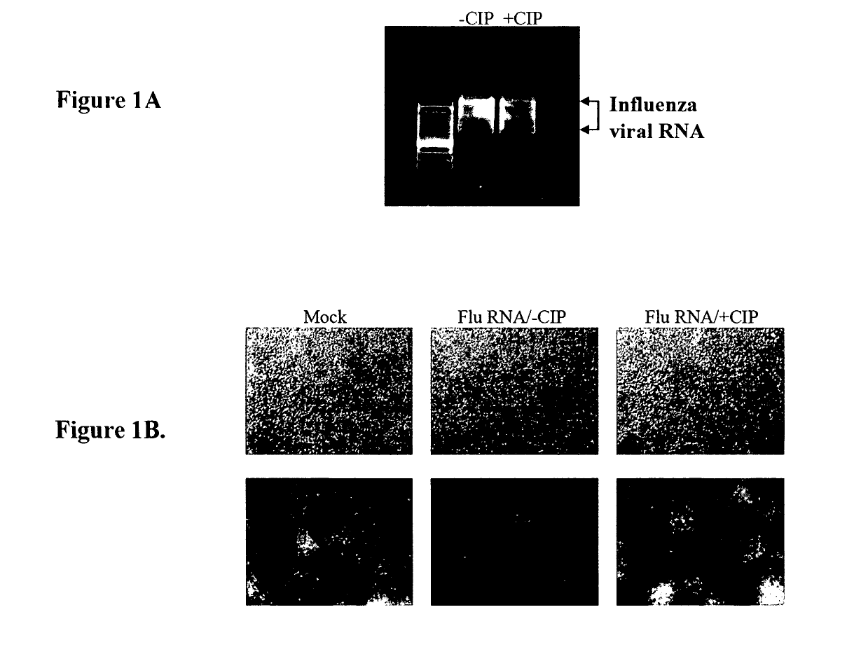

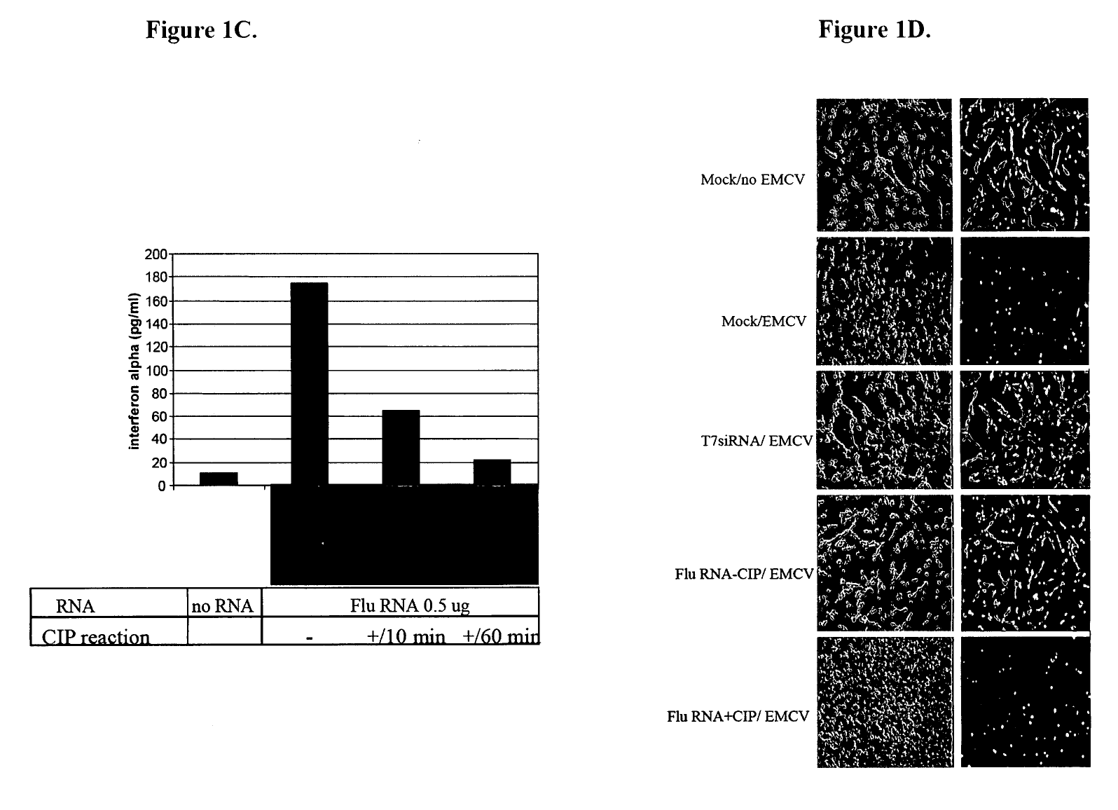

Another group has also found that ssRNA transcribed by the T7 phage polymerase is a potent interferon inducer and proposed that a structural RNA or dsRNA motif of mRNA is an endogenous ligand for the receptor³³. The present findings which indicate that the nuclear RNA, but not cytoplasmic RNA, activates the interferon response (FIG. 2C) is contradictory to their claims. The same group showed that capped mRNA activates the interferon response. Since the complete capping reaction of used mRNA was not confirmed in that experiment, it is possible that the innate immunity is still activated by residual amount of uncapped RNA in the reaction. If any dsRNA motif present on nascent RNAs is capable of inducing the interferon response, cells will be faced with a serious threat whereby any self ssRNA can represent a danger signal. The lost of innate immunity by the CIP treated influenza viral RNA (FIGS. 1B and 1D), CIP treated nuclear RNA (FIG. 2C), and CIP treated RNA* (FIGS. 5B and 6C) clearly indicate that the 5′ triphosphate is an initiator of interferon response. Another group has shown that monocytes can be differentiated into antigen-loading mature dendritic cells by introducing in vitro transcribed mRNA³⁴. Based on these studies, it is speculated that the stimulatory effect of mRNA is through the activation of the innate immune response mediated by the 5′ triphosphate motif.

The data support a model where two RNA ligands are activated through a common receptor (FIGS. 6B and 6C). Previous work has shown the involvement of TLR3 in lung epithelial cells, where TLR3 is induced by dsRNA and influenza A virus infection³⁵. This appears to parallel the data where influenza viral RNA induces interferons in a TLR3 dependent manner (FIG. 6C). Most strikingly this study has shown that TLR3 recognizes both dsRNA as well as the 5′ triphosphate-containing RNA (FIGS. 6B and 6C). It would be of great interest to observe how the same receptor can distinguish between the two different ligands and to establish the unique profile of each pathway. Understanding the specific attributes of the 5′ tri-phosphate mediated pathway would shed light on defining a new mechanism for potential anti-viral responses. Recognition of ssRNAs derived from Vesicular Stomatosis Virus (VSV) and influenza virus through TLR7 in dendritic and B cells has been previously reported⁷. Additionally, influenza viral RNA and some synthetic RNA such as poly U RNA can induce the interferon response through TLR7 and 8 in dendritic cells and macrophages^{5,6,8}. It was found that poly U RNA cannot induce innate immune responses in HeLa, HEK293, and NIH3T3 cells (data not presented). Additionally, TLR3 recognizes influenza viral RNA in human fibroblast cells³¹. One explanation for the activity of different TLRs against influenza viral RNA may be owing to cell type specificity. It is possible that cells adopt a different mechanism for a more diverse and coordinated innate response against pathogenic invasion, perhaps by using different receptors with limited localization.

Additional screening found RNA* is up to 100 fold more potent in the anti-viral response than the ssRNA with the 5′triphosphate. Since the RNA* induces a more potent, longer lasting and less toxic antiviral effect than poly IC, it may potentially be used as a new anti-viral reagent. The reaction is mediated rolling circle transcription with a special rule (Table 5). The transcription is started from 3′ to 5′ direction of ssDNA and repeated several times to make monomer to multimers (Table 5). Interestingly the first three nucleotides of 3′ end are not transcribed in the monomer. However they are faithfully transcribed in all multimers. Since the fourth nucleotide is the initiation site of transcription the first three nucleotides may serve a binding site of the polymerase. Small circled DNA mediated rolling circle transcription has been reported 32. However the exact initiation site and nature of transcribed DNA template have not been identified. With understanding of the exact mechanism of transcription using linear ssDNAs, it would be feasible to make several RNA products such as catalytic RNAs, apatamers, and siRNAs with less expense and labor.

RNA*

In one aspect of the invention, an advantageous feature of the invention is the use of transcripts obtained using in vitro rolling circle mechanism of transcription. An inventive aspect relates to use of single stranded DNA template, which may be absent a promoter sequence, in which the template is about 19 to about 60 nucleotides long, preferably 24 to about 45 nucleotides long, is able to be transcribed in vitro using standard transcription reagents that are conventionally used in the industry. Such transcription kits include without limitation, ones sold at Ambion, Inc., which is exemplified in the present application.

DNA Template

Single stranded DNA template is used in this reaction. Without being limited by theory, it is believed that in order for the rolling circle mechanism of transcription to be efficiently carried out, the DNA template must form a secondary structure that is not too “tight” such as a hairpin structure as shown in Table 4, nor too “loose” such as the noose structure shown also at lower, left of Table 4. A DNA sequence that folds into a secondary structure of a partial loop or partial foldback appears to be the more effective template for making the 5′-triphosphate concatemers of the invention.

In addition to the structural aspects of the template DNA, the length of the template oligonucleotide DNA may also be considered. The oligonucleotide may be without limitation about 19 to about 60 nucleotides long, preferably, about 19 to about 50 nucleotides, more preferably 24 to about 45 nucleotides long. In addition, the oligonucleotide may be optionally free of a promoter sequence.

RNA Concatemer

The length of the RNA concatemer that is made using the rolling circle transcription reaction may be a monomer, dimer, trimer, tetramer, and so forth, without limitation. The transcripts generated are mixtures of concatemers and monomers. For instance, if a 24 nucleotide single stranded DNA is used as a template, the first reaction cycle results in a RNA monomer having 5′ triphosphate that is 21 nucleotides long, and subsequent second cycle adds 24 nucleotides so that the dimer is 45 nucleotides long, and the third cycle results in a trimer having 69 nucleotides and so forth as 24 nucleotide segments are added to raise the concatenation by a monomer after each subsequent cycle.

Contacting cells with the mixture of 5′-triphosphate concatemers results in at least 100 fold greater anti-viral activity and induction of innate immune response, as well as inducing apoptosis in cancer cells, and preventing proliferation in non-tumorous cells.

Administration

The present invention can be practiced in vitro or in vivo. The invention also can be used as a therapeutic or preventative agent, preferably for therapy or prevention of a disease or condition.

An effective amount refers to that amount of RNA effective to produce the intended result, including the intended pharmacological, therapeutic or preventive result. In cell culture, an effective amount for initiating an antiviral effect can be as low as 1 nM, and can range up to 20 nM or more. However, it is understood that higher dosages can be toxic to cells, due to unregulated induction, resulting in undesired levels of expression of several cytokines, including interferon. A pharmaceutically effective amount or dose is that amount or dose required to prevent, inhibit the occurrence, or treat (alleviate a symptom to some extent, preferably all of the symptoms) of a disease state. The pharmaceutically effective amount or dose depends on the type of disease, the composition use, the route of administration, the type of mammal being treated, the physical characteristics of the specific mammal under consideration, concurrent medication, and other factors which those skilled in the medical arts will recognize. Generally, an effective amount or dose of dsRNA or ssRNA for human use is known in the art and/or can be determined by standard methods, and can be administered, for example, in the ranges of about 0.001 mg/kg to 100 mg/kg body weight/day or about 0.01 mg/kg to 10 mg/kg body weight/day.

Methods for formulating compositions and reagents in accordance with the invention, as well as modes of administration, are known in the art and are described, for example, in U.S. Pat. Nos. 6,645,943 and 6,001,992, which are incorporated herein by reference in their entirety. Formulations can include a pharmaceutically or physiologically acceptable carrier, such as an inert diluent or an assimilable edible carrier. The pharmaceutical compositions of the present invention may be administered in a number of ways depending upon whether local or systemic treatment is desired and upon the area to be treated. Administration may be topical (including ophthalmic and to mucous membranes including vaginal and rectal delivery), pulmonary, e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Coated condoms, gloves and the like may also be useful. Compositions and formulations for oral administration include powders or granules, suspensions or solutions in water or non-aqueous media, capsules, sachets or tablets. Thickeners, flavoring agents, diluents, emulsifiers, dispersing aids or binders may be desirable.

Methods for delivering the RNA molecules of the invention into cells also are well known in the art such as disclosed in U.S. Pat. No. 6,673,611, which is incorporated herein by reference in its entirety. RNA may be directly introduced into the cell (i.e., intracellularly); or introduced extracellularly into a cavity, interstitial space, into the circulation of an organism, introduced orally, or may be introduced by bathing an organism in a solution containing the RNA. Methods for oral introduction include direct mixing of the RNA with food of the organism, as well as engineered approaches in which a species that are used as food is engineered to express the RNA, then fed to the organism to be affected. Physical methods of introducing nucleic acids, for example, injection directly into the cell or extracellular injection into the organism, may also be used. Vascular or extravascular circulation, the blood or lymph system, the phloem, the roots, and the cerebrospinal fluid are sites where the RNA may be introduced.

Physical methods of introducing nucleic acids include injection of a solution containing the RNA, bombardment by particles covered by the RNA, soaking the cell or organism in a solution of the RNA, or electroporation of cell membranes in the presence of the RNA. A viral construct packaged into a viral particle would accomplish both efficient introduction of an expression construct into the cell and transcription of RNA encoded by the expression construct. Other methods known in the art for introducing nucleic acids to cells may be used, such as lipid-mediated carrier transport, chemical-mediated transport, such as calcium phosphate, and the like. Thus the RNA may be introduced along with components that perform one or more of the following activities: enhance RNA uptake by the cell, promote annealing of the duplex strands, stabilize the annealed strands, or other-wise increase inhibition of the target gene.

Methods for the delivery of nucleic acid molecules also are described in Akhtar and Juliano, Trends Cell Biol, 2, 139 (1992) and Akhtar, Delivery Strategies for Antisense Oligonucleotide Therapeutics, CRC Press, Boca Raton, Fla. (1995), each of which is incorporated herein by reference. These protocols can be utilized for the delivery of virtually any nucleic acid molecule. Nucleic acid molecules can be administered to cells by a variety of methods known to those familiar to the art, including, but not restricted to, encapsulation in liposomes, by iontophoresis, or by incorporation into other vehicles, such as hydrogels, cyclodextrins, biodegradable nanocapsules, and bioadhesive microspheres. Alternatively, the nucleic acid/vehicle combination is locally delivered by direct injection or by use of an infusion pump. Other routes of delivery include, but are not limited to oral (tablet or pill form), intrathecal, mucosal, or transdermal delivery. Other approaches include the use of various transport and carrier systems, for example, through the use of conjugates and biodegradable polymers.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims. The following examples are offered by way of illustration of the present invention, and not by way of limitation.

### EXAMPLES

### Example 1

### Materials and Methods

Reagents

Poly IC was purchased from Sigma. Poly IC was further purified through extraction twice with phenol followed by ethanol precipitation. For determination of interferon alpha and beta, ELISA kits were purchased from RDI (Concord, Mass.). HEK293, NIH3T3, and MRC5 cells were cultured in DMEM media supplemented with 10% Fetus Bovine Serum and glutamine. The enolase antibody was purchased from Biogenesis (Kingston, HN). HnRNP H antibody was a generous gift from Dr. Black Lab at UCLA (Los Angeles, Calif.). Cytoplasmic and nuclear extracts were prepared as described with a modification 36. The isolated nuclei and cytoplasmic extract were mixed with Stat 60 (Tel-Test) followed by the Manufacturer's instructions to purify RNA. For cloning of EGFP genes into pBluescipt vector the Not1 and BamH1 fragment of pDNR-EGFP vector (Clonetech) was interested to the Not1 and BamH1 site. The short EGFP fragment was made after deletion of Sal1 fragment.

SiRNAs

The anti-poliovirus siRNA (siC²⁹; sense sequence 5′ GCGUGUAAUGACUUCAGCGUG 3′ (SEQ ID NO:1) was synthesized by the oligo synthesis facility at the City of Hope (Duarte, Calif.). Anti-TLR3 siRNA with the form of dicer substrate siRNA^{38,39} (sense sequence 5′ GGUAUAGCCAGCUAACUAGCUUGGA (SEQ ID NO:2), and antisense sequence 5′ UCCAAGCUAGUUAGCUGGCUAUACCUU (SEQ ID NO:3)) were synthesized from Samchully Pharm. Co. (Seoul, Korea).

RNA Transcription

All short RNAs were synthesized using the Silencer siRNA Construction kit from Ambion, Inc. according to the manufacturer's protocol. To transcribe T7 RNA in vitro, T7 primer I (5′ TAATACGACTCACTATA 3′ (SEQ ID NO:4)) was hybridized with T7 primer II which contains the antisense sequence of each transcribed RNA and the tail sequence: 5′ CCCTATAGTGAGTCGTA 3′ (SEQ ID NO:5). The hybridized oligos were used for Klenow reaction. For RNA* synthesis, each indicated DNA oligo set forth in the Tables was used as template for the transcription reaction using the Silence siRNA construction kit. Each RNA* was purified using the column included in the kit. To determine optimum concentration of Mg²⁺, the transcription buffer in the kit was replaced with regular reaction buffer (40 mM Tris-HCl (pH 7.5), 6 mM MgCl₂, 10 mM NaCl, 2 mM spermidine) with increased concentration of Mg²⁺. For the CIP treatment, 20 U of enzyme (NEB) was added to each RNA and further incubated at 37° C. for 1 hour or the indicated time. DNA templates used in Table 4 are as follows:

(SEQ ID NO:6)

5′-CAG GTA GCA GCC GAC ACC GGT CTG GCT TCC ATC
ATG-3′,

(SEQ ID NO:7)

5′-CAC TCA TTC TGA GGC GTC GAC TGA CAG GTC TTC
AGT-3′,

(SEQ ID NO:8)

5′-TAA TAC GAC TCA CTA TAG GGG AAG GAC ATT GAA GGA
CAA A-3′,

(SEQ ID NO:9)

5′-TTT GTC CTT CAA TGT CCT TCC CCT ATA GTG AGT CGT
ATT A-3′,

(SEQ ID NO:10)

5′-CAT GAT GGA AGC CAG ACC GGT CTG GCT TCC ATC
ATG-3′.

Transfection

All transfection assays were done using Lipofectamine 2000 (Invitrogen). The cells at 90 percent confluency were transfected with each RNA of indicated amount. The split cells were diluted into 50% confluency using the culture media and directly mixed with the preformed complex containing the fixed amount of RNA in 50 ul of PBS and 1 ul of lipofectamine. The complex was incubated for 15 minutes and directly added on the top of culture media.

Viral Challenge Assay

For anti-HSV-1 or anti-polioviral assays, 60% confluent 293 cells on plates were transfected with RNA using Lipofectamine 2000 (Invitrogen). The following day (24 hours) the cells were infected with HSV-1 expressing the EGFP or Poliovirus Mahoney strain at a multiplicity of infection of 1 or 0.1, respectively. 24 hours post infection, the anti-HSV activity was measured by determining the EGFP level in the extract using a Fluorometer (Bio-Rad). To prepare the extract, the cells in the 24 well plates were mixed with 200 μl of passive lysis buffer (Promega). For the anti-polioviral assay, the cells in each well were washed with PBS three times to remove dead cells caused by the cytotoxic effect of the virus and lysed by adding 200 μl of the lysis buffer. Total amount of protein was measured by Bradford assay. For anti-proliferation effect of RNA* or poly IC, 40% of the NIH3T3 cells stably expressing EGFP gene was plated in 24 well plates on day 1. The cells were transfected with RNA* or poly IC and harvested on day 5. Total cell numbers were determined by measuring EGFP levels using the fluorometer. For the anti-EMCV activity assay, cells were transfected with indicated amount of each RNA on day 2. On day 3, the cells were infected with a 0.1 MOI of EMCV. Total anti-EMCV activity was measured on day 5 or day 7. Total numbers of survived cells were determined by the level of EGFP expression in the extracts after normalization to the value of each parallel sample from the anti-proliferation activity assay that was not challenged by the virus.

Viral RNA

Influenza virus A/PR/8/34 (PR8), subtype H1N1, a kind gift from Dr. Peter Palese, Mount Sinai School of Medicine, was grown for 48 hours in 10-day-embryonated chicken eggs (Charles River laboratories, MA) at 37° C. 48 hours after virus inoculation, the allantoic fluid was harvested and was centrifuged at 1300 rpm for 10 min. The supernatant was then mixed with 25% sucrose in 0.1 M Tris (pH 8.0) and ultracentrifuged at 25,000 rpm for 2 hours using SW28. Following centrifugation, Trizol (Invitrogen) was added to the pellet and RNA purification was performed according to the manufacturer's instructions.

RT-PCR

Total RNA was purified using Stat60 and treated with 2 U of DNase (Promega) per ug of RNA for 20 min at 37° C. To detect Tlr3, Tlr7 and β-actin mRNA using reverse transcriptase (RT) and PCR, first strand cDNA synthesis was performed at 37° C. for 1 hour in a 30 μl reaction mixture containing 2 μg of total cellular RNA, 2 μmol of gene-specific primer (50 ng of random primers (Invitrogen)), 0.5 mM each of dATP, dCTP, dTTP and dGTP, 3 mM MgCl₂, 50 mM Tris-HCl (pH 8.3), 75 mM KCl, 20 mM DTT, 5 U RNasin RNase inhibitor (Promega) and 200 U M-MLV Reverse Transcriptase (Invitrogen). Reverse primers used for the PCR reaction (see below) were used as gene-specific primers for first strand synthesis of Tlr3 and Tlr7. Aliquots (5 μl) of the cDNA reaction mixture were used to amplify Tlr3, Tlr7 and P-actin sequences separately. The PCR reaction mixtures included 50 mM KCl, 10 mM Tris-HCl (pH 8.3 at 25° C.), 1.5 mM Mg(OAc)₂, 0.2 mM each of dATP, dCTP, dTTP and dGTP, 15 μmol each of forward and reverse primers, and 2.5 U of Taq DNA polymerase (Eppendorf). Sequences of forward and reverse Tlr3 primers were

5′ AGATACAACGTAGCTGACTGCAGCCATTTG 3′ (SEQ ID NO:11) and 5′ CTTCACTTCGCAACGCAAGGATTTTATTTT 3′ (SEQ ID NO:12). Sequences of forward and reverse Tlr7 primers were 5′CATTCCCACTAACACCACCAATCTTACCCT 3′ (SEQ ID NO:13) and 5′ ATCCTGTGGTATCTCCAGAAGTTGGTTTCC 3′ (SEQ ID NO:14). Sequences of forward and reverse β-actin primers were 5′ ACCAACTGGGACGACATGGAGAAGATCTGG 3′ (SEQ ID NO: 15) and 5′ GCTGGGGTGTTGAAGGTCTCAAACATGATC 3′ (SEQ ID NO: 16). Thermal cycling reactions were conducted at 95° C. for 30 seconds, 58° C. for 30 seconds and 72° C. for 1 minute. Aliquots were removed from the PCR reaction mixtures during the exponential phase of amplification after 25 (β-actin) and 35 cycles (Tlr3 and Tlr7). Samples were resolved using 2% agarose gel electrophoresis.

### Example 2

### Identification of RNA* Sequencing Using RACE

In vitro transcribed RNA* was separated on 8% denaturing polyacryamide gel and two RNA bands about 60˜70, and 80˜90 size were excised. Excised band is incubated at 37° C. for 3 hours to elute RNA. Eluted RNA is phenol-chloroform extracted and ethanol precipitated. To identify RNA* sequence, RACE (SMART-RACE kit, Clontech) was performed according to the manufacturer's instructions with some modifications. One ug of RNA was poly(A) tailed using poly(A) polymerase (USB, United States Biochemical) according to the manufacturer's instructions. Poly(A) tailed RNA was purified with Sephadex G-25 (Sigma) column and followed by RACE reaction. 5′, 3′ RACE reactions were performed in one tube simultaneously, because sequence information about RNA* was not available. 3′ RACE CDS primer A (for 3′ RACE) and SMARTII™ A oligonucleotide (for 5′ RACE) were added to 1 ug of poly(A) tailed RNA in 5 μl. An additional 5 μl, containing the rest of the reaction components were added to the mixture on ice, and the reactions were incubated at 42° C. for 1.5 hrs. SMARTII™ A oligo 5′-AAGCAGTGGTATCAACGCAGAGTACGCGGG-3′ (SEQ ID NO:17) hybridized to the 3′ end of the cDNA (5′ end of RNA) during the reverse transcription reaction. PowerScript reverse transcriptase adds several untemplated C residues to the 3′ end of cDNA, and the template switching mechanism (SMART) enables reverse transcription from the SMARTII™ A oligo which adds its complement to the 3′ end of cDNA. 30 cycles of PCR were performed on the samples using 10× Universal primer A mix (UPM) and 5′-GAGTAC(T)₃₀-3′ primer as gene specific primer. RACE PCR products were eluted from the agarose gel, cloned using pGEM T-easy vector system (Promega) and sequenced using ABI PRISM™ 310 genetic analyzer.

### Example 3

### Functional Assay for TLR3

The functional inhibition assay was followed as previously described³⁹. Anti-TLR2 and TLR3 antibodies were purchased from eBioscience (San Diego, Calif.). Briefly, MRC-5 cells in 24 well plates (1×10⁵ cells) were pre-incubated with 20 μg/ml of anti-TLR2 or anti-TLR3 antibody for 1 hour at 37° C. The cells were transfected with either 5 ng of RNA* or 250 ng of poly IC. The next day, interferon beta levels in the media were determined by ELISA (RDI). siRNA mediated TLR3 suppression assay was carried out as described³¹. The sequence of 21mer siRNA was converted to Dicer substrated siRNA as shown elsewhere³⁸. Each cell was transfected with 20 nM of either scrambled or anti-TLR3 siRNA for 48 hours. Sequentially, the cells were transfected with 5 ng of RNA* or 250 ng of poly IC. After 24 hours, the cells were infected with 0.1 MOI of HSV. The viability of cells was monitored using MTA assay in 24 hour.

### Example 4

### Microarray

RNA was isolated applying an RNeasy Mini kit (Qiagen GmbH, Hilden, Germany), according to the manufacturer's instructions. Or total RNA was extracted using TRIzol (Invitrogen, Carlsbad, Calif.) and the RNeasy Mini kit (Qiagen, Valencia, Calif.)). The purity and integrity of the total RNA were checked using NanoDrop (NanoDrop Technologies) and Experion (Biorad), respectively.

Mouse oligonucleotides were purchased from Operon Technologies Inc. (Alameda, Calif.) and Sigma-Genosys (The Woodlands, Tex.), and were inkjet-printed by Agilent Technologies (Palo Alto, Calif.). The 16K oligo array includes 13,536 Operon designed and synthesized probes (70mer), and 2,304 Compugen Ltd. (Jamesburg, N.J.) designed and Sigma-Genosys synthesized probes (65mer). The aminoallyl method was used for the preparation of fluorescently labeled target samples. Briefly, both first and second strand cDNAs were synthesized by incubating 3 μg of total RNA with the T7 promoter primer (5′-GGCCAGTGAATTGTAATACGACTCACTATAGGGAGGCGG-(dT)₂₄-3′ (SEQ ID NO: 18)) (Qiagen Inc., Valencia, Calif.) followed by using SuperScript II (Invitrogen Life Technologies, Carlsbad, Calif.). Aminoallyl-UTP (aaUTP) labeled antisense RNA (aRNA) was synthesized by adding reagents to the 15 μl of cDNA template in the following order: 4 μl of 75 mM ATP solution; 4 μl of 75 mM CTP solution; 4 μl of 75 mM GTP solution; 2 μl of 75 mM UTP solution; 4 μl of 10× reaction buffer; 3 μl of 50 mM aaUTP (Ambion, Austin, Tex.); and 4 μl of MEGAscript T7 enzyme mix (Ambion). The coupling reaction was performed by mixing 10 μg of aRNA with 2 μl of 0.5 M sodium bicarbonate, pH 9.5, along with 10 μl of mono-Cy3 or mono-Cy5 solution (PerkinElmer, Inc.; Boston, Mass.), and adjusting the final volume to 20 μl/reaction. Three μg of each labeled aRNA target was hybridized after being fragmented by mixing with fragmentation buffer (Agilent Technologies). After hybridization and washing, oligo arrays were scanned by the Agilent Scanner G2505A (Agilent Technologies). Genes that were saturated, non-uniform, or not significantly above background (below 2.6× standard deviation of background) in either channel were removed.

For human chip, probe synthesis from total RNA samples, hybridization, detection, and scanning were performed according to standard protocols from Affymetrix, Inc. The data was analyzed using GeneChip Operating Software (Affymetrix, Calif.). Only relative changes equal or greater than 2 fold level of expression were considered.

### Example 5

### Identification of Effecter Molecules in the Cultured Media

To define the anti-viral or anti-proliferation activity of RNA*, 50 ul of media was collected from the 293 cells transfected with the RNA after three days later and sequentially incubated with 2 ul of individual antibody serum for 30 min at room temperature. In anti-viral assay the antibody neutralized media was directly added to the 293 cells in 24-well plate. The antibody against interferon beta was purchased from PBL (product number 21450-1). After 24 hours HSV-GFP virus was added to the culture by 0.1 MOI. The level of GFP which is the indicator of viral infection was monitored in 48 hours. For the anti-proliferation assay the media was incubated in the presence or absence of antibody and mixed to MDA-MB-468 cell culture which has been plated in 25% density. The viability of cells was determined by MTT assay in 3 days.

### Example 6

### Cloning and Production of Interferon Beta

Human interferon beta gene was cloned using two PCR primers using total human genome. The PCR product was digested with Sal1 and Age1 enzyme and cloned to Sal1 and Age1 site of plegfp-C1 vector. 293 cells were transfected with the vector and cultured in the media containing G418 to select the individual clones. The anti-viral or anti-proliferation activity was tested using the media cultured from each clones. A clone shows the best activity was selected for further production of interferon beta.

All of the references cited herein are incorporated by reference in their entirety.

Those skilled in the art will recognize, or be able to ascertain using no more than routine experimentation, many equivalents to the specific embodiments of the invention specifically described herein. Such equivalents are intended to be encompassed in the scope of the claims.

### CITED REFERENCES

1. Boehme, K. W. & Compton, T. Innate sensing of viruses by toll-like receptors. J Virol 78, 7867-73 (2004).
2. Beutler, B. Innate immunity: an overview. Mol Immunol 40, 845-59 (2004).
3. Malmgaard, L. Induction and regulation of IFNs during viral infections. J Interferon Cytokine Res 24, 439-54 (2004).
4. Alexopoulou, L., Holt, A. C., Medzhitov, R. & Flavell, R. A. Recognition of double-stranded RNA and activation of NF-kappaB by Toll-like receptor 3. Nature 413, 732-8 (2001).
5. Hornung, V. et al. Sequence-specific potent induction of IFN-alpha by short interfering RNA in plasmacytoid dendritic cells through TLR7. Nat Med 11, 263-70 (2005).
6. Heil, F. et al. Species-specific recognition of single-stranded RNA via toll-like receptor 7 and 8. Science 303, 1526-9 (2004).
7. Lund, J. M. et al. Recognition of single-stranded RNA viruses by Toll-like receptor 7. Proc Natl Acad Sci USA 101, 5598-603 (2004).
8. Diebold, S. S., Kaisho, T., Hemmi, H., Akira, S. & Reis e Sousa, C. Innate antiviral responses by means of TLR7-mediated recognition of single-stranded RNA. Science 303, 1529-31 (2004).
9. Kim, D. H. et al. Interferon induction by siRNAs and ssRNAs synthesized by phage polymerase. Nat Biotechnol 22, 321-5 (2004).
10. Lamb, R. A. & Choppin, P. W. The gene structure and replication of influenza virus. Annu Rev Biochem 52, 467-506 (1983).
11. Elliott, G. & O'Hare, P. Live-cell analysis of a green fluorescent protein-tagged herpes simplex virus infection. J Virol 73, 4110-9 (1999).
12. Kim, D. H. et al. Synthetic dsRNA Dicer substrates enhance RNAi potency and efficacy. Nat Biotechnol 23, 222-6 (2005).
13. Pichlmair, A. et al. RIG-I-Mediated Antiviral Responses to Single-Stranded RNA Bearing 5′ Phosphates. Science (2006).
14. Hornung, V. et al. 5′-Triphosphate RNA Is the Ligand for RIG-I. Science (2006).
15. Gu, M. & Lima, C. D. Processing the message: structural insights into capping and decapping mRNA. Curr Opin Struct Biol 15, 99-106 (2005).
16. Wei, C. M., Gershowitz, A. & Moss, B. 5′-Terminal and internal methylated nucleotide sequences in HeLa cell mRNA. Biochemistry 15, 397-401 (1976).
17. Chou, M. Y., Rooke, N., Turck, C. W. & Black, D. L. hnRNP H is a component of a splicing enhancer complex that activates a c-src alternative exon in neuronal cells. Mol Cell Biol 19, 69-77 (1999).
18. Dolken, G., Leisner, E. & Pette, D. Immunofluorescent localization of glycogenolytic and glycolytic enzyme proteins and of malate dehydrogenase isozymes in cross-striated skeletal muscle and heart of the rabbit. Histochemistry 43, 113-21 (1975).
19. Geiss, G. et al. A comprehensive view of regulation of gene expression by double-stranded RNA-mediated cell signaling. J Biol Chem 276, 30178-82 (2001).
20. Saravolac, E. G. et al. Immunoprophylactic strategies against respiratory influenza virus infection. Vaccine 19, 2227-32 (2001).
21. Katze, M. G. The war against the interferon-induced dsRNA-activated protein kinase: can viruses win? J Interferon Res 12, 241-8 (1992).
22. Levine, A. S. & Levy, H. B. Phase I-II trials of poly IC stabilized with poly-L-lysine. Cancer Treat Rep 62, 1907-12 (1978).
23. Cohen, S. A., Tzung, S. P., Doerr, R. J. & Goldrosen, M. H. Role of asialo-GM1 positive liver cells from athymic nude or polyinosinic-polycytidylic acid-treated mice in suppressing colon-derived experimental hepatic metastasis. Cancer Res 50, 1834-40 (1990).
24. Chacko, M. S., Ma, X. & Adamo, M. L. Double-stranded ribonucleic acid decreases c6 rat glioma cell proliferation in part by activating protein kinase R and decreasing insulin-like growth factor I levels. Endocrinology 143, 2144-54 (2002).
25. Lampkin, B. C., Levine, A. S., Levy, H., Krivit, W. & Hammond, D. Phase II trial of poly(I,C)-LC, an interferon inducer, in the treatment of children with acute leukemia and neuroblastoma: a report from the Children's Cancer Study Group. J Biol Response Mod 4, 531-7 (1985).
26. Heitmeier, M. R., Scarim, A. L. & Corbett, J. A. Double-stranded RNA inhibits beta-cell function and induces islet damage by stimulating beta-cell production of nitric oxide. J Biol Chem 274, 12531-6 (1999).
27. Moriyama, H. et al. Induction and acceleration of insulitis/diabetes in mice with a viral mimic (polyinosinic-polycytidylic acid) and an insulin self-peptide. Proc Natl Acad Sci USA 99, 5539-44 (2002).
28. Andino, R. RNAi puts a lid on virus replication. Nat Biotechnol 21, 629-30 (2003).
29. Gitlin, L., Karelsky, S. & Andino, R. Short interfering RNA confers intracellular antiviral immunity in human cells. Nature 418, 430-4 (2002).
30. Beutler, B. Inferences, questions and possibilities in Toll-like receptor signalling. Nature 430, 257-63 (2004).
31. Salaun, B., Coste, I., Rissoan, M. C., Lebecque, S. J. & Renno, T. TLR3 can directly trigger apoptosis in human cancer cells. J Immunol 176, 4894-901 (2006).
32. Daubendiek, S. L. & Kool, E. T. Generation of catalytic RNAs by rolling transcription of synthetic DNA nanocircles. Nat Biotechnol 15, 273-7 (1997).
33. Kariko, K., Ni, H., Capodici, J., Lamphier, M. & Weissman, D. mRNA is an endogenous ligand for Toll-like receptor 3. J Biol Chem 279, 12542-50 (2004).
34. Ponsaerts, P. et al. Messenger RNA electroporation is highly efficient in mouse embryonic stem cells: successful FLPe- and Cre-mediated recombination. Gene Ther 11, 1606-10 (2004).
35. Guillot, L. et al. Involvement of toll-like receptor 3 in the immune response of lung epithelial cells to double-stranded RNA and influenza A virus. J Biol Chem 280, 5571-80 (2005).
36. Robb, G. B., Brown, K. M., Khurana, J. & Rana, T. M. Specific and potent RNAi in the nucleus of human cells. Nat Struct Mol Biol 12, 133-7 (2005).
37. Bhuyan, P. K. et al. Short interfering RNA-mediated inhibition of herpes simplex virus type I gene expression and function during infection of human keratinocytes. J Virol 78, 10276-81 (2004).
38. Rose, S. D. et al. Functional polarity is introduced by Dicer processing of short substrate RNAs. Nucleic Acids Res 33, 4140-56 (2005).
39. Matsumoto, M., Kikkawa, S., Kohase, M., Miyake, K. & Seya, T. Establishment of a monoclonal antibody against human Toll-like receptor 3 that blocks double-stranded RNA-mediated signaling. Biochem Biophys Res Commun 293, 1364-9 (2002).

What is claimed is:

1. A method of making RNA comprising, mixing a single stranded DNA template with a RNA transcription reagent and allowing transcription reaction to occur.

2. The method according to claim 1, wherein the DNA is about 19 to about 60 nucleotides long.

3. The method according to claim 2, wherein the DNA is about 24 to about 45 nucleotides long.

4. The method according to claim 1, wherein the DNA is absent a promoter sequence.

5. The method according to claim 1, wherein the DNA has a partial-loop secondary structure, which allows for rolling circle type transcription.

6. The method according to claim 1, wherein the RNA has a length of at least 21 nucleotides.

7. The method according to claim 6, wherein the RNA is concatenated.

8. A method for inducing production of interferon in a cell, comprising introducing into the cell a composition comprising an effective amount of 5′-triphosphate concatenated RNA, wherein the RNA molecule induces the interferon.

9. The method of claim 8, wherein the RNA molecule induces interferon-beta.

10. The method of claim 8, wherein the RNA molecule also has an anti-viral effect.

11. The method of claim 8, wherein the RNA molecule is introduced into the cell prior to viral infection.

12. The method of claim 8, wherein the RNA molecule inhibits viral infection.

13. An isolated 5′-triphosphate RNA molecule having a length of at least 21 nucleotides, made according to the method of claim 1.

14. The isolated RNA according to claim 13, wherein the molecule is concatenated.

15. A method of inhibiting viral infection in a mammal comprising administering to the mammal a composition comprising an effective amount of the RNA molecule according to claim 14, which elicits an anti-viral response.

16. A method of killing tumor cells through apoptosis in a mammal, comprising administering to the mammal a composition comprising an effective amount of the RNA molecule according to claim 14.

17. A method of inhibiting proliferation of cancerous cells in a mammal, comprising administering to the mammal a composition comprising an effective amount of the RNA molecule according to claim 14, wherein when the RNA molecule enters a normal cell, interferon-beta is secreted, and inhibits proliferation of nearby tumor cells.

18. A method of activating innate immune response in a mammal, comprising administering to the mammal a composition comprising an effective amount of the RNA molecule according to claim 14.


SEQUENCE LISTING (23 sequences)

SEQ ID NO: 1 - RNA, 21 nt, Artificial
  siRNA
gcguguaaugacuucagcgug

SEQ ID NO: 2 - RNA, 25 nt, Artificial
  siRNA
gguauagccagcuaacuagcuugga

SEQ ID NO: 3 - RNA, 27 nt, Artificial
  siRNA
uccaagcuaguuagcuggcuauaccuu

SEQ ID NO: 4 - DNA, 17 bp, Artificial
  Primer
taatacgactcactata

SEQ ID NO: 5 - DNA, 17 bp, Artificial
  Primer
ccctatagtgagtcgta

SEQ ID NO: 6 - DNA, 36 bp, Artificial
  Oligo
caggtagcagccgacaccggtctggcttccatcatg

SEQ ID NO: 7 - DNA, 36 bp, Artificial
  Oligo
cactcattctgaggcgtcgactgacaggtcttcagt

SEQ ID NO: 8 - DNA, 40 bp, Artificial
  Oligo
taatacgactcactataggggaaggacattgaaggacaaa

SEQ ID NO: 9 - DNA, 40 bp, Artificial
  Oligo
tttgtccttcaatgtccttcccctatagtgagtcgtatta

SEQ ID NO: 10 - DNA, 36 bp, Artificial
  Oligo
catgatggaagccagaccggtctggcttccatcatg

SEQ ID NO: 11 - DNA, 30 bp, Artificial
  Primer
agatacaacgtagctgactgcagccatttg

SEQ ID NO: 12 - DNA, 30 bp, Artificial
  Primer
cttcacttcgcaacgcaaggattttatttt

SEQ ID NO: 13 - DNA, 30 bp, Artificial
  Primer
cattcccactaacaccaccaatcttaccct

SEQ ID NO: 14 - DNA, 30 bp, Artificial
  Primer
atcctgtggtatctccagaagttggtttcc

SEQ ID NO: 15 - DNA, 30 bp, Artificial
  Primer
accaactgggacgacatggagaagatctgg

SEQ ID NO: 16 - DNA, 30 bp, Artificial
  Primer
gctggggtgttgaaggtctcaaacatgatc

SEQ ID NO: 17 - DNA, 30 bp, Artificial
  Oligo
aagcagtggtatcaacgcagagtacgcggg

SEQ ID NO: 18 - DNA, 39 bp, Artificial
  Primer
ggccagtgaattgtaatacgactcactatagggaggcgg

SEQ ID NO: 19 - DNA, 36 bp, Artificial
  Oligo
taatacgactcactatagctaaagtaggtagtgtta

SEQ ID NO: 20 - DNA, 36 bp, Artificial
  Oligo
taacactacctactttagctatagtgagtcgtatta

SEQ ID NO: 21 - DNA, 17 bp, Artificial
  Oligo
tatagtgagtcgtatta

SEQ ID NO: 22 - DNA, 36 bp, Artificial
  Oligo
attatgctgagtgatatcgatttcatccatcacaat

SEQ ID NO: 23 - DNA, 19 bp, Artificial
  Oligo
taacactacctactttagc